SEQUENCE LISTING (19 sequences)

SEQ ID NO: 1 - protein, 214 aa, Artificial Sequence
  trastuzumab LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial Sequence
  trastuzumab HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  pertuzumab LC
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, Artificial Sequence
  pertuzumab HC
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence
  common light chain
DIQMTQSPSSLSASVGDRVTITCKASQDVSTAVAWYQQKPGKAPKLLIYSASFRYTGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, Artificial Sequence
  trastuzumab HC knob
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 449 aa, Artificial Sequence
  pertuzumab HC hole
EVQLVESGGGLVQPGGSLRLSCAASGFTFNDYTMDWVRQAPGKGLEWVADVNPNSGGSIVNRRFKGRFTLSVDRSKNTLYLQMNSLRAEDTAVYYCARNLGPFFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 450 aa, Artificial Sequence
  CrossMab HC1
QVQLVQSGAEVKKPGASVKVSCKASGFNIKDTYIHWVRQAPGQGLEWMGRIYPTNGYTRYAQKFQGRVTMTRDTSISTAYMELSRLRSDDTAVYYCSRWGGEGFYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 453 aa, Artificial Sequence
  CrossMab HC2
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  CrossMab LC1
DIQLTQPPSVSVAPGQTARITCGASQDVSTAVAWYQQKPGQAPVLVVYSASFLYSGIPSRFSGSRSGTDFTLTISRVEAGDEADYYCQQHYTTPPTFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 212 aa, Artificial Sequence
  CrossMab LC2
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - RNA, 19 nt, Artificial Sequence
  siRNA CD46-1
gagagagcacgauuuauug

SEQ ID NO: 13 - RNA, 19 nt, Artificial Sequence
  siRNA CD46-2
caauaaaucgugcucucuc

SEQ ID NO: 14 - RNA, 19 nt, Artificial Sequence
  siRNA CD55-1
gaagaguucugcaaucgua

SEQ ID NO: 15 - RNA, 19 nt, Artificial Sequence
  siRNA CD55-2
uacgauugcagaacucuuc

SEQ ID NO: 16 - RNA, 19 nt, Artificial Sequence
  siRNA CD59-1
gcaagaaggaccuguguaa

SEQ ID NO: 17 - RNA, 19 nt, Artificial Sequence
  siRNA CD59-2
uuacacagguccuucuugc

SEQ ID NO: 18 - RNA, 19 nt, Artificial Sequence
  scrambled siRNA-1
ugcagguuuauaguccaca

SEQ ID NO: 19 - RNA, 19 nt, Artificial Sequence
  scrambled siRNA-2
uguggacuauaaaccugca

(19) United States
(12) Patent Application Publication
(10) Pub. No.: US 2019/0241661 A1
(43) Pub. Date: Aug. 8, 2019

(54) ASSAY AND METHOD FOR DETERMINING CDC ELICITING ANTIBODIES

(71) Applicant: Hoffmann-La Roche Inc., Little Falls, NJ (US)

(72) Inventors: Stefan Dengl, Muenchen (DE); Sonja Offner, Penzberg (DE)

(73) Assignee: Hoffmann-La Roche Inc., Little Falls, NJ (US)

(21) Appl. No.: 16/218,804

(22) Filed: Dec. 13, 2018

Related U.S. Application Data
(63) Continuation of application No. PCT/EP2017/064272, filed on Jun. 12, 2017.

(30) Foreign Application Priority Data
Jun. 16, 2016 (EP) 16174675.5

(51) Int. Cl. C07K 16/28; A61K 47/68; C07K 14/47; G01N 33/50

(52) U.S. Cl. CPC C07K 16/2818; A61K 47/68; C07K 16/2896; C07K 2317/24; G01N 33/5014; G01N 2500/10; C07K 2317/734; C07K 14/472

(57) ABSTRACT

Herein is reported a method for determining complement dependent cytotoxicity of a composition comprising i) a first binding site that specifically binds to a first epitope on a first antigen, which is conjugated to a first Fc-region polypeptide of human origin, and ii) a second binding site that specifically binds to a second epitope on a second antigen, which is conjugated to a second Fc-region polypeptide of human origin, wherein the method comprises the steps of incubating a cell expressing the first antigen and the second antigen with the composition and a mixture of anti-mCRP antibodies; adding normal human serum or rabbit complement to the mixture; and determining cell lysis and thereby determining complement dependent cytotoxicity of the composition.

### CROSS-REFERENCE TO RELATED APPLICATIONS

This application is a continuation of International Patent Application No. PCT/EP2017/064272, having an international filing date of Jun. 12, 2017, the entire contents of which are incorporated herein by reference, and which claims benefit under 35 U.S.C. § 119 to European Patent Application No. 16174675.5 filed Jun. 16, 2016

### SEQUENCE LISTING

The instant application contains a Sequence Listing submitted via EFS-Web and hereby incorporated by reference in its entirety. Said ASCII copy, created on Dec. 12, 2018, is named P33684-US_Sequence Listing.txt, and is 35,085 bytes in size.

### FIELD OF THE INVENTION

The current invention is in the field of assays and methods for the detection/selection of effector function eliciting antibodies and antibody combinations.

### BACKGROUND

Immunoglobulins contain two binding sites for certain Fc receptors, such as FcRn, as well as for C1q, one in each heavy chain Fc-region.

For complement activation more than a single immunoglobulin molecule is required as the affinity of monomeric IgG for C1q is quite weak (affinity about 10⁻⁴ M) (see e.g. Sledge et al., J. Biol. Chem. 248 (1973) 2818-2813, Hughes-Jones et al., Mol. Immunol. 16 (1979) 697-701). The binding of the multivalent C1q may be increased by antigen-based association of the immunoglobulin molecules and, thus, complement activation (affinity about 10⁻⁸ M) (see e.g. Burton et al., Mol. Immunol. 22 (1990) 161-206).

The three dimensional structure of C1q is like a bunch of tulips comprising six globular heads, which comprise the antibody binding regions (see e.g. Perkins et al., Biochem. J. 228 (1985) 13-26, Poon et al., J. Mol. Biol. 168 (1983) 563-577, Reid et al., Biochem. Soc. Trans. 11 (1983) 1-12, and Weiss et al., J. Mol. Biol. 189 (1986) 573-581).

In U.S. Pat. No. 5,851,528 are reported methods of inhibiting complement activation. Recombinant antibodies against CD55 and CD59 and uses thereof are reported in U.S. Pat. No. 8,034,902. In US 2012/0226020 hybrid and chimeric polypeptides that regulate activation of complement are reported. Novel modulators and methods of use are reported in US 2013/0302355. In US 2010/0255011 compositions and methods for modulating the activity of complement regulatory proteins on target cells are reported.

In WO 2008/007648 it is reported that classifying antibody, involves contacting antibody capable of recognizing cell surface antigen with cell of same species, analyzing each cell and comparing obtained data and classifying individual antibodies depending on similarity. Compositions and methods for modulating the activity of complement regulatory proteins on target cells are reported in WO 2010/120541.

Mekhaiel, D.N.A., et al, report polymeric human Fc-fusion proteins with modified effector functions (Nature Sci. Rep. 1 (2011) 1-11). Polypeptide variants with altered effector function are reported in WO 00/42072. In US 2008/0089892 Fc region variants are reported. Altered antibody Fc regions and uses thereof are reported in WO 2006/105062.

Neonatal rabbit complement was used to deplete lymphocytes from different complex immune cell populations with the help of antibodies to facilitate transplantation (see e.g. Herve, P., et al., Transplant. 39 (1985) 138-143).

Baby Rabbit complement was not successful in eliciting complement dependent cytotoxicity (CDC) in renal cell carcinoma (RCC) using antibodies of murine origin (see e.g. Vessella, R. L., et al., Canc. Res. 45 (1985) 6131-6139).

Rabbit serum could kill human SK-Mel28 melanoma cells (non-epithelial=non-carcinoma) by CDC using single and paired murine IgG2a antibodies binding p97 (=melanotransferrin) (see e.g. Hellstroem, I., et al., Int. J. Canc. 31 (1983) 553-555).

Membrane-bound complement regulatory proteins (mCRPs) have a lower expression level on lymphocytes compared to monocytes and neutrophils (see e.g. Nuutila, J., et al., Hum. Immunol. 74 (2013) 522-530).

The up-regulation of mCRPs as an immune escape mechanism is more pronounced on most of the cancer cells than e.g. on lymphomas or melanomas (see e.g. Fishelson, Z., et al., Mol. Immunol. 40 (2003) 109-123).

Antibodies were used to show CDC either in settings with syngeneic serum (e.g. normal human serum (NETS) together with human carcinoma cells and human antibodies) without the CDC-inhibitory influence of mCRPs (see e.g. Dechant et al., 2008, Cancer Research) or with syngeneic serum (e.g. normal human serum (NETS) together with human carcinoma cells and human antibodies) showing a strong mCRP dependent CDC-inhibitory effect that had to be overcome by the siRNA-dependent down regulation of the mCRPs CD46, CD55 and CD59 (see e.g. Mamidi, S., et al., Mol. Onc.7 (2013) 580-594).

Konishi, e., et al. reported the utilization of complement-dependent cytotoxicity to measure low levels of antibodies: application to nonstructural protein 1 in a model of Japanese encephalitis virus (Clin. Vac. Immunol. 15 (2008) 88-94). Klitgaard, J., et al. reported that the combination of two anti-cos monoclonal antibodies synergistically induces complement-dependent cytotoxicity of chronic lymphocytic leukemia cells (Brit. J. Hematol. 163 (2013) 182-193). Hellstrom, I., et al. reported that monoclonal antibodies to two determinants of melanoma-antigen p97 act synergistically in complement-dependent cytotoxicity (J. Immunol. 127 (1981) 157-160). Maddipatla, S., et al., reported augmented antitumor activity against B-cell lymphoma by a combination of monoclonal antibodies targeting Trail-R1 and CD20 (Clin. Cancer Res. 13 (2007) 4556-4564). Huang, J., et al. reported about the protection of xenogeneic cells from human complement-mediated lysis by the expression of human DAF, CD59 and MCP (FEMS Immunol. Med. Microbiol. 31 (2001) 203-209. Qu, Z., et al. reported about recombinant bispecific monoclonal antibody (bsmAb) against CD20 and CD22 active in vitro and in vivo against B-cell lymphomas (Blood 108 (2006) 713a-714a). Hellstrom et al. have reported that cell-mediated suppression of tumor immunity has a non-specific component (Int. J. Cancer 27 (1981) 481-485 and 487-491).

AU 2011/202520 discloses human monoclonal antibodies against CD20. WO 2016/096788 discloses assay and method for determining CDC eliciting antibodies. US 2006/0035267 discloses optimal polyvalent vaccine for cancer. Guo, B., et al. (Clin. Immunol. 128 (2008) 155-163) discloses mapping of binding epitopes of a human decay-accelerating factor monoclonal antibody capable of enhancing rituximab-mediated complement-dependent cytotoxicity.

### SUMMARY

Herein is reported an improved assay for the determination and analysis of the CDC capacity regarding carcinoma cells of carcinoma-cell surface antigen binding antibodies. This assay does not require tedious, complicated and instable approaches, such as e.g. siRNA down-regulation of mCRPs (membrane-bound complement regulatory proteins). The current approach counteracts the up-regulation of mCRPs in carcinoma cells (per definition these are of epithelial origin) as immune escape mechanism for evading the CDC pressure in the body by the addition of a combination of anti-mCRP antibodies. In contrast to epithelial cancer cells this is not a major response in lymphoid tumor cells. The current assay provides a means to determine CDC of carcinoma-cell surface antigen binding antibodies that cannot elicit CDC in other settings due to the effect of the mCRPs.

It has been found that normal human serum together with human or humanized antibodies and human carcinoma cells can be used to elicit complement dependent cytotoxicity (CDC) in human cells, especially human carcinoma cells, in a very robust manner if mCRP up-regulation in said cells is counteracted by the addition of a combination of anti-mCRP antibodies. By using anti-mCRP antibodies in combination with normal human serum for the determination of the CDC capacity of human or humanized antibodies specifically binding to carcinoma cell surface antigens
the up-regulated human mCRPs on carcinoma cells do not abrogate the CDC-eliciting effect of human or humanized antibodies as observed in other assay setups,
the unreliability that in some cases normal human serum (NETS) could only elicit CDC with human or humanized antibodies and human tumor cells, if the mCRPs were down-regulated by siRNA, could be overcome, and
high throughput screening of the CDC capacity of different antibodies, antibody formats or antibody conjugates is now possible.

The method as reported herein can be used with tumor cells, such as lymphoma cells (lymphoma=lymphoid tumor=lymphocytic origin) or carcinoma cells (carcinoma=epithelial origin), as well as cell eliciting an autoimmune response.

One aspect as reported herein is a method for determining complement dependent cytotoxicity of a composition comprising i) a first binding site that specifically binds to a first epitope on a first antigen, which is conjugated to a first Fc-region polypeptide of human origin, and ii) a second binding site that specifically binds to a second epitope on a second antigen, which is conjugated to a second Fc-region polypeptide of human origin, wherein the method comprises the following steps:
a) incubating a cell expressing the first antigen and the second antigen with the composition and a mixture of anti-mCRP antibodies,
b) adding normal human serum or rabbit complement to the mixture of a), and
c) determining cell lysis and thereby determining complement dependent cytotoxicity of the composition.

One aspect as reported herein is a method for selecting a composition comprising i) a first binding site that specifically binds to a first epitope on a first antigen, which is conjugated to a first Fc-region polypeptide of human origin, and ii) a second binding site that specifically binds to a second epitope on a second antigen, which is conjugated to a second Fc-region polypeptide of human origin that has CDC-activity, wherein the method comprises the following steps:
a) incubating individually a cell expressing the first antigen and the second antigen with two or more of said compositions and a mixture of anti-mCRP antibodies,
b) adding normal human serum or rabbit complement to the mixture of a),
c) determining cell lysis and thereby determining complement dependent cytotoxicity of said compositions, and
d) selecting based on the result of step c) a composition that has CDC-activity.

One aspect as reported herein is a method for determining complement dependent cytotoxicity of an antibody comprising i) (at least) a first binding site that specifically binds to a first epitope on a first antigen, ii) optionally a second binding site that specifically binds to a second epitope on a second antigen, wherein the method comprises the following steps:
a) incubating a cell expressing (at least) the first antigen and optionally the second antigen with the antibody and a mixture of anti-mCRP antibodies,
b) adding normal human serum or rabbit complement to the mixture of a), and
c) determining cell lysis and thereby determining complement dependent cytotoxicity of the antibody.

One aspect as reported herein is a method for overcoming species specific mCRP-induced inhibition of complement dependent cytotoxicity of an antibody comprising i) (at least) a first binding site that specifically binds to a first epitope on a first antigen, ii) optionally a second binding site that specifically binds to a second epitope on a second antigen, wherein the method comprises the following steps:
a) incubating a cell expressing (at least) the first antigen and optionally the second antigen with the antibody and a mixture of anti-mCRP antibodies,
b) adding normal human serum or rabbit complement to the mixture of a), and
c) determining cell lysis and thereby determining complement dependent cytotoxicity of the antibody.

In one preferred embodiment of all aspects the mixture of anti-mCRP antibodies is a mixture comprising an anti-CD46 antibody, an anti-CD55 antibody and an anti-CD59 antibody.

In one preferred embodiment of all aspects the mixture is added in a tenfold saturating amount.

In one preferred embodiment the anti-mCRP antibodies are added at a 10-times saturating concentration, whereby the 1-times saturating concentration is defined as the concentration of the antibodies as determined by FACS analyses that is (just) sufficient for a saturated staining of the cells. In one embodiment the dilution showing in a FACS device upon incubation with a (single) cell (a fluorescence signal close to) the (obtained) maximum fluorescence signal is the 1-time saturating concentration.

In one embodiment the anti-mCRP antibodies have a non-human Fc-region. In one embodiment the anti-mCRP antibodies have a murine Fc-region.

In one embodiment of all aspects the antibody is an antibody format.

In one embodiment of all aspects the two or more compositions differ in the first and/or second epitope or antigen.

In one embodiment of all aspects the composition comprises a first human or humanized antibody that specifically binds to a first epitope on a first antigen and a second human or humanized antibody that specifically binds to a second epitope on a second antigen.

In one embodiment of all aspects the composition comprises a human or humanized bispecific antibody that specifically binds to a first epitope on a first antigen and a second epitope on a second antigen.

In one embodiment of all aspects the first antigen and the second antigen are the same antigen and the first epitope and the second epitope are different. In one embodiment the first epitope and the second epitope are non-overlapping epitopes.

In one embodiment of all aspects cell lysis is determined between 0.5 and 3 hours after the addition of complement or normal human serum.

In one embodiment of all aspects the cell is a cancer cell. In one embodiment the human cell is a human cancer cell. In one embodiment the cancer cell is a carcinoma cell. In one preferred embodiment the cancer cell is a carcinoma cell of epithelial origin.

In one embodiment the human carcinoma cell of epithelial origin is selected from the group consisting of human ovary adenocarcinoma cells, and human breast adenocarcinoma cells. In one preferred embodiment the human carcinoma cell of epithelial origin is selected from a SK-OV3 cell, and a MCF7 cell.

In one embodiment of all aspects the rabbit complement is Baby Rabbit complement.

In one embodiment of all aspects the ratio of the first binding site to the second binding site is of from 10:1 to 1:10. In one embodiment the ratio is of from 0.5:1 to 1:0.5.

### BRIEF DESCRIPTION OF THE DRAWINGS

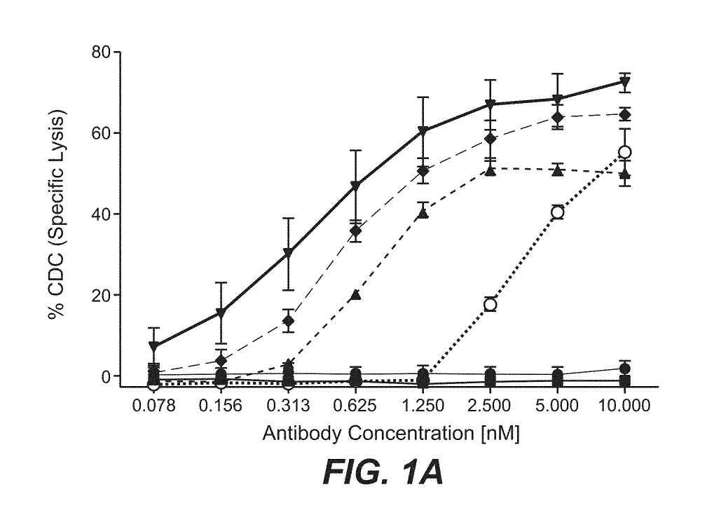

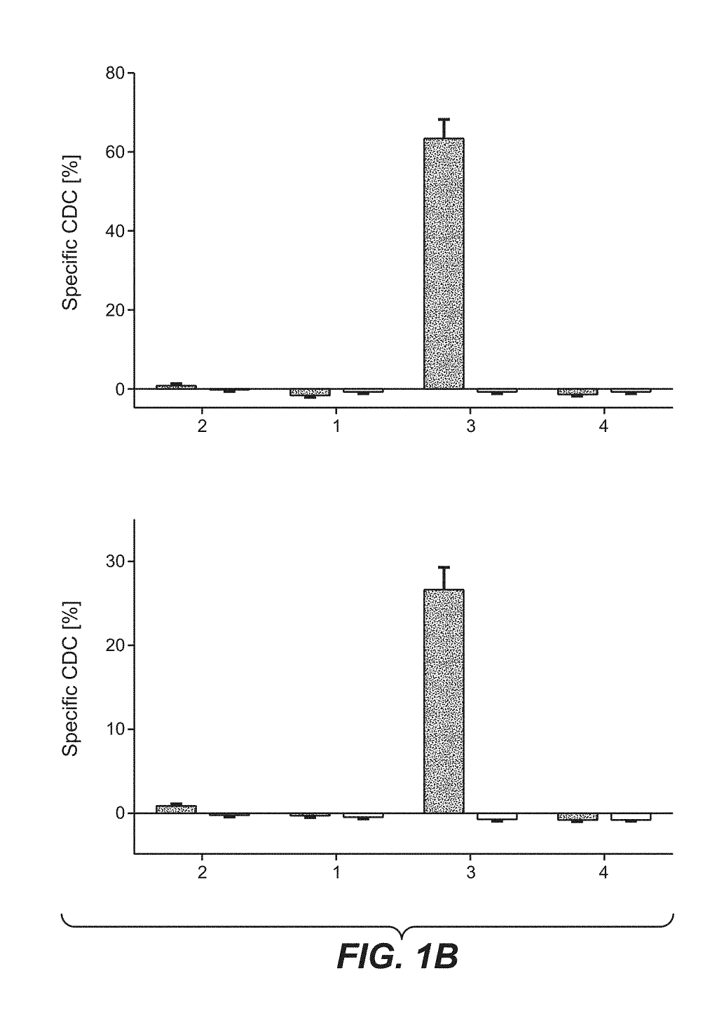

### FIG. 1A and FIG. 1B:

FIG. 1A: Specific CDC on BT-474 cells determined by LDH release and shown as % CDC; closed circles: trastuzumab; closed squares: pertuzumab; upward triangle: combination of trastuzumab and pertuzumab; downward triangle=bispecific anti-HER2 antibody, common light chain; diamond=bispecific anti-HER2 antibody, common light chain, glycoengineered; open circle=bispecific anti-HER2 antibody, CrossMab format.

FIG. B: Specific CDC on BT-474 cells (upper graph) and SK-Br3 cells (lower graph); 1=trastuzumab; 2=pertuzumab; 3=combination of trastuzumab and pertuzumab; 4=human IgG1, kappa light chain control; left bars: specific CDC with Baby Rabbit complement; right bars: specific CDC without Baby Rabbit complement; % CDC and specific CDC means specific cytotoxicity [%].

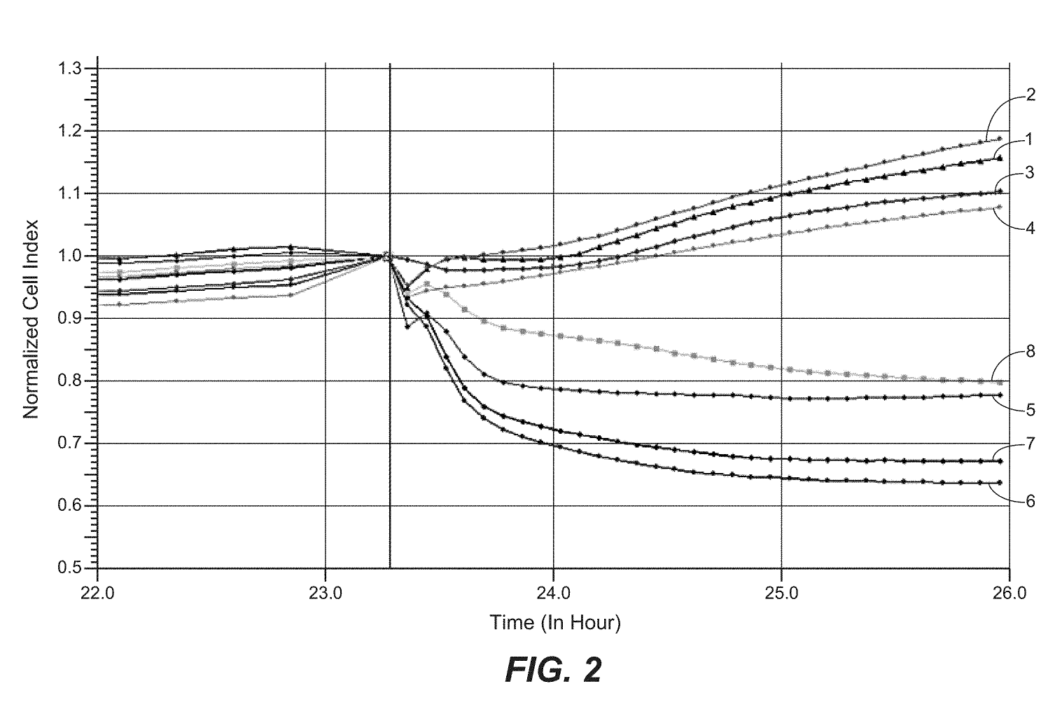

FIG. 2: Time course of cell index (ACEA); 1=trastuzumab; 2=pertuzumab; 3=medium only; 4=complement control; 5=combination of trastuzumab and Pertuzumab; 6=bispecific anti-HER2 antibody, common light chain; 7=bispecific anti-HER2 antibody, common light chain, glycoengineered; 8=bispecific anti-HER2 antibody, CrossMab format.

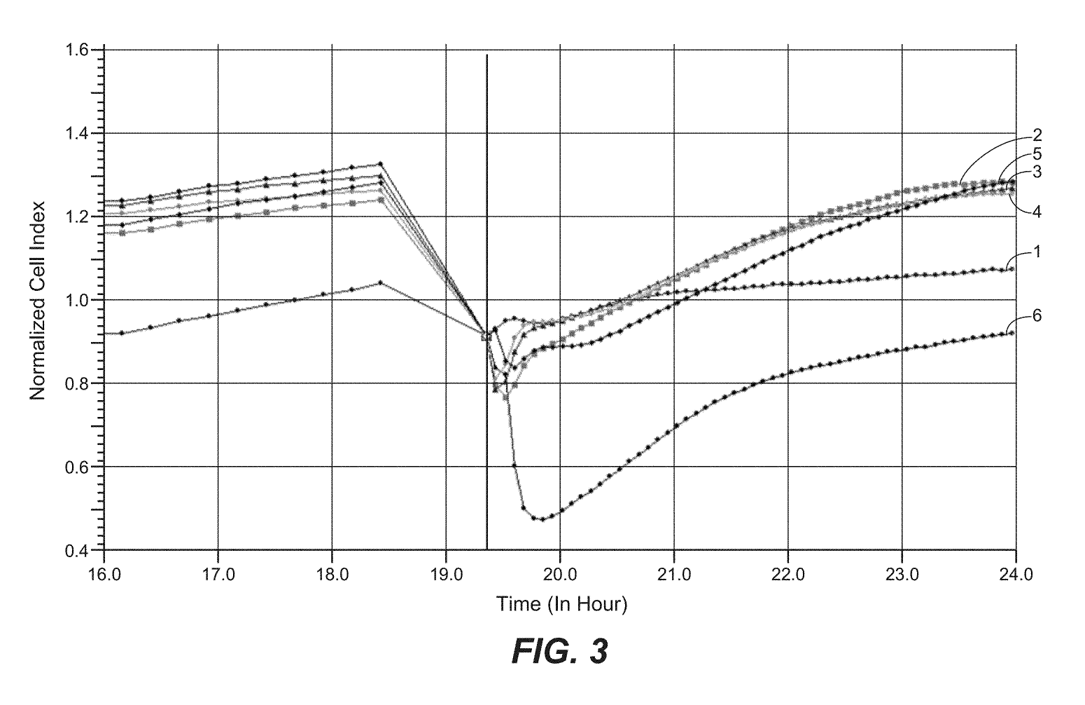

FIG. 3: Time course of cell index (ACEA); 1=medium only; 2=complement control; 3=with anti-CD55 antibody, human serum pool, trastuzumab, pertuzumab; 4=with anti-CD59 antibody, human serum pool, trastuzumab, pertuzumab; 5=with anti-CD55 antibody, anti-CD59 antibody, human serum pool, trastuzumab and pertuzumab; 6=trastuzumab, pertuzumab and Baby Rabbit complement.

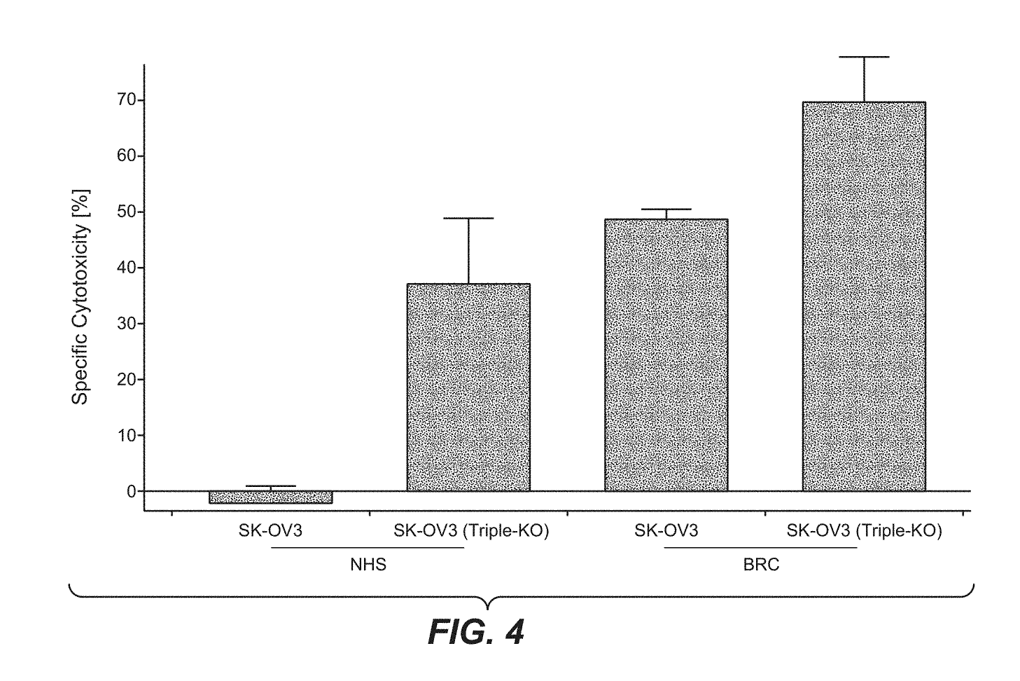

FIG. 4: Results of the CDC assay using CD46, CD55, CD59 knockdown (triple-KO) SK-OV-3 cells. Cells were incubated with 10 μg/mL antibody each, Baby Rabbit complement and Normal Human Serum respectively.

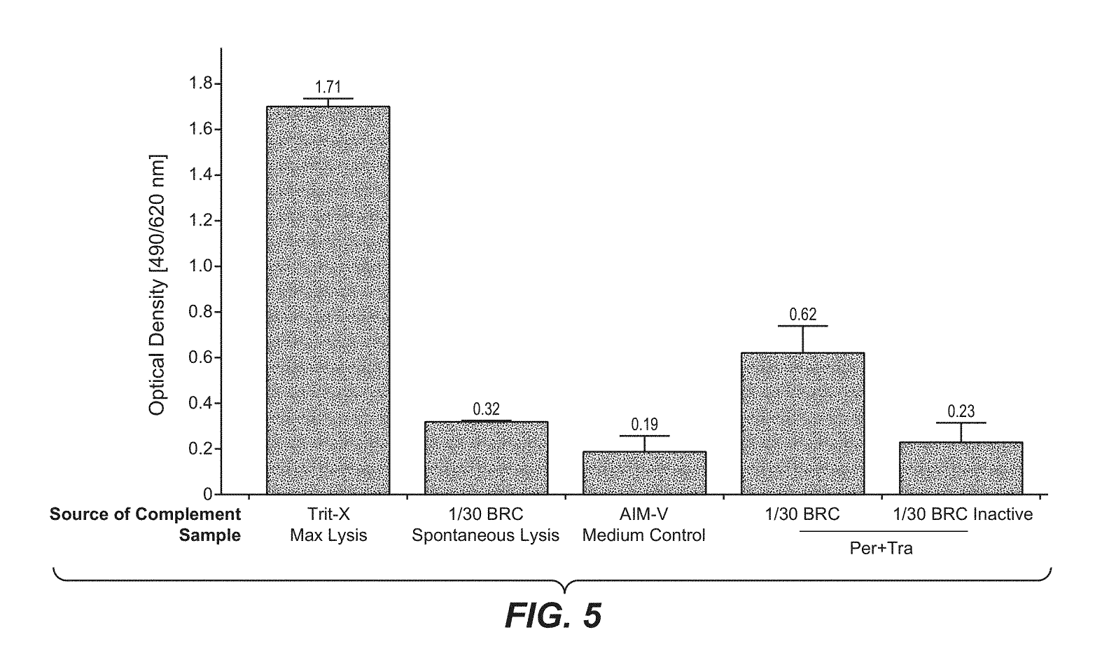

FIG. 5: Results of a BRC CDC-assay using as readout optical density. The x-axis depicts different samples (Max. lysis, spontaneous lysis, medium control, 10 μg/ml Per+Tra in combination with active and inactive 1/30 BRC). The bar values and standard deviation were calculated from triplicates.

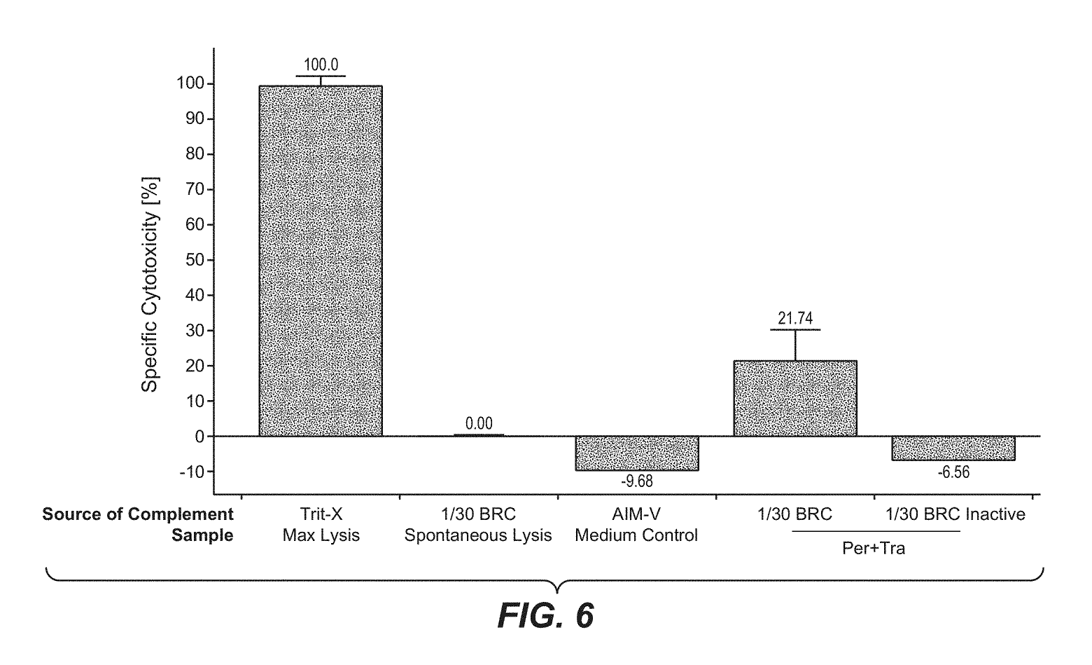

FIG. 6: Results of a BRC CDC-assay after conversion to specific cytotoxicity. The x-axis depicts different samples (Max. lysis, spontaneous lysis, medium control, 10 μg/ml Per+Tra in combination with active and inactive 1/30 BRC). The bar values and standard deviation were calculated from triplicates. Max. lysis is always set to 100% and spontaneous lysis is always set to 0%.

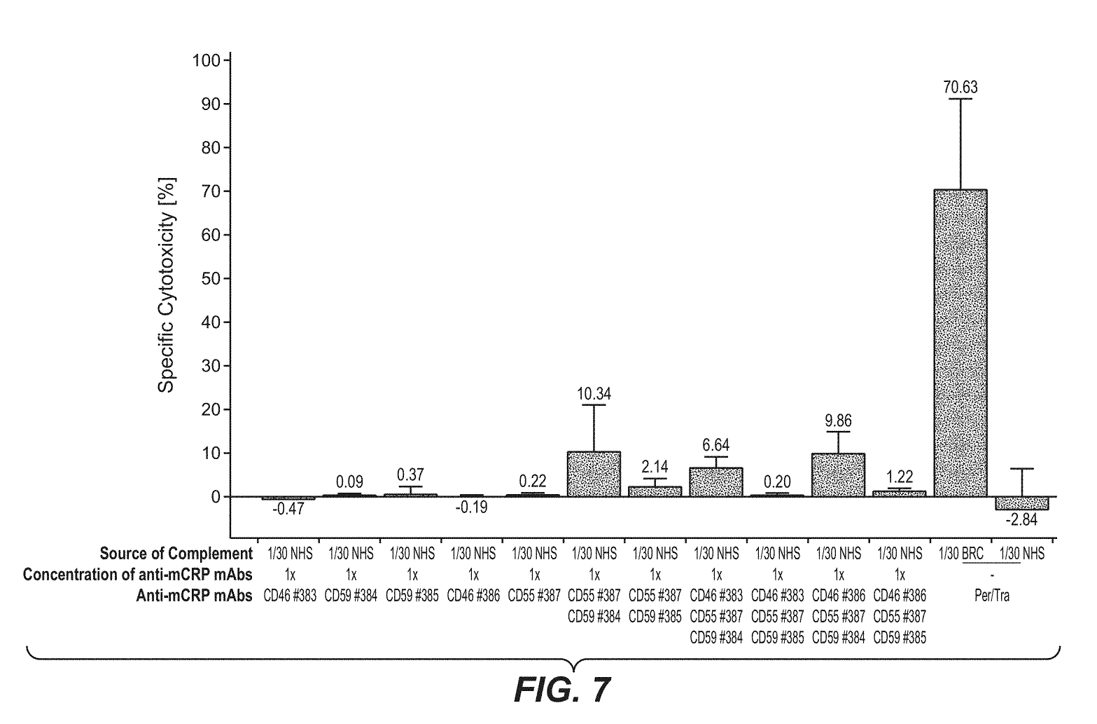

FIG. 7: Results of NHS CDC-assay with different combinations of anti-mCRP blocking mAbs using a 1-times saturating concentration. The x-axis describes the source of complement, the concentration of anti-mCRP mAbs (1×), as well as the compilation of the anti-mCRP mAbs used to block the mCRPs. The concentration of Per+Tra was 10 μg/ml. NHS (1/30) and BRC (1/30) were used as source of complement. The bar values and standard deviation were calculated from triplicates.

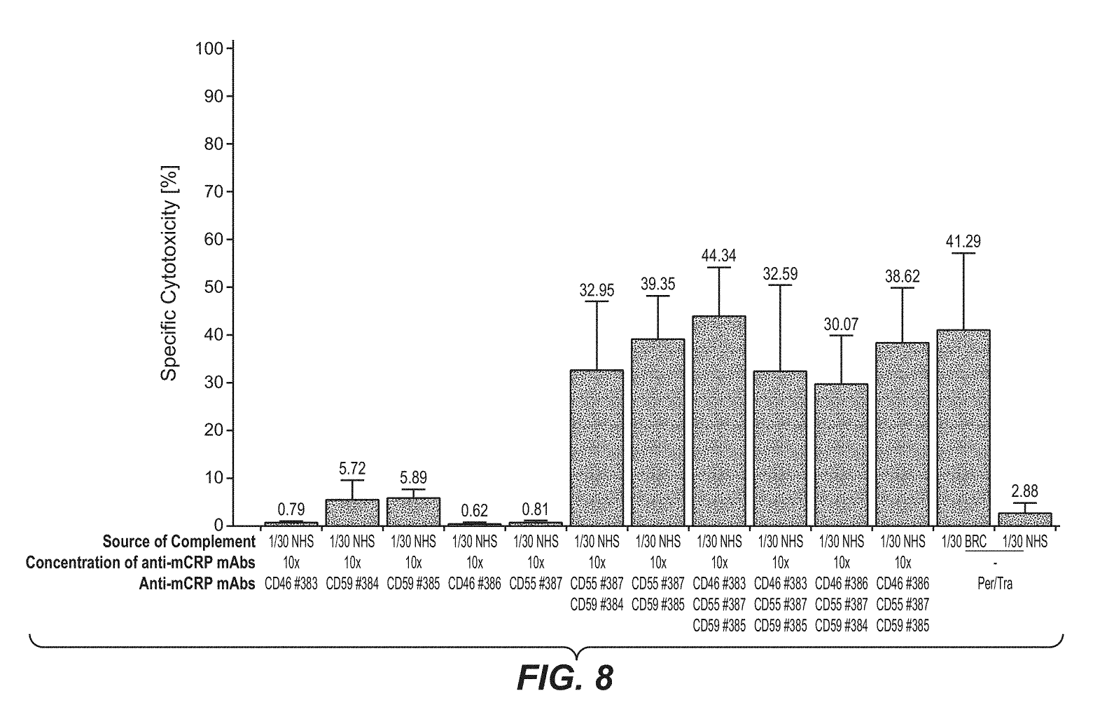

FIG. 8: Results of NHS CDC-assay with different combinations of anti-mCRP blocking mAbs using a tenfold saturating concentration. The x-axis describes the source of complement, the concentration of anti-mCRP mAbs (10×), as well as the compilation of the anti-mCRP mAbs used to block the mCRPs. The concentration of Per+Tra was 10 μg/ml. NHS (1/30) and BRC (1/30) were used as source of complement. The bar values and standard deviation were calculated from triplicates.

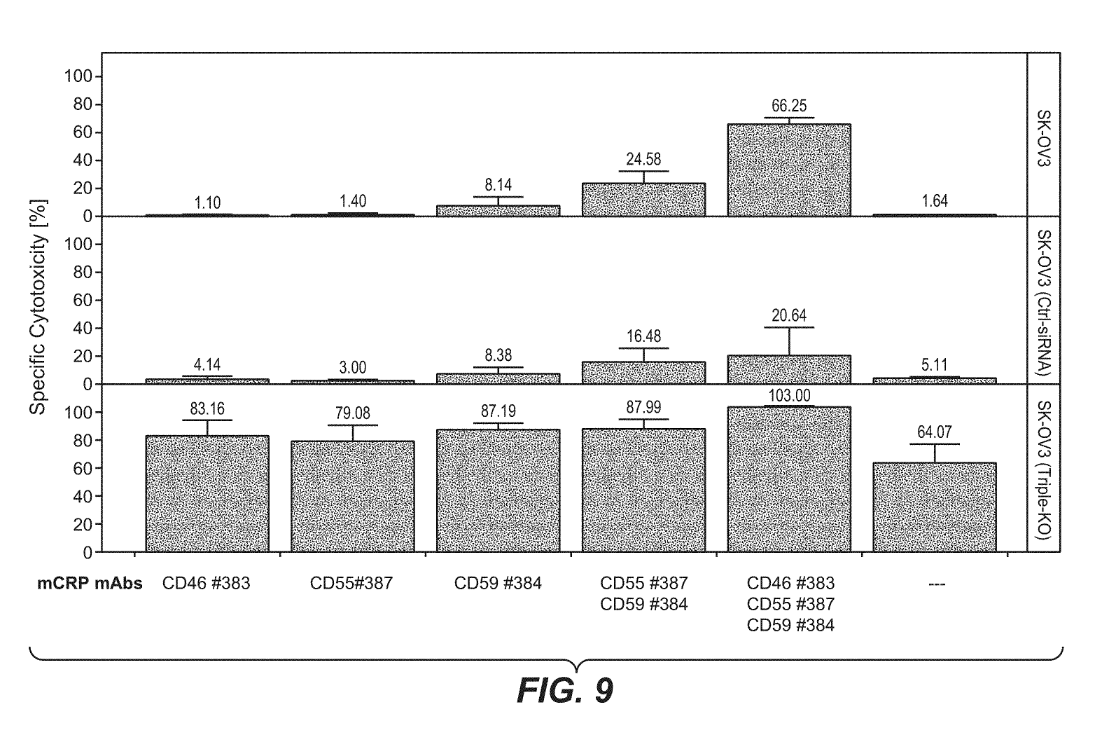

FIG. 9: Results of NHS CDC-assay with Per+Tra. The antibodies Per+Tra were used at 10 μg/ml. The human complement (NETS) was used in a 1/30 dilution. The 10-times saturating concentration of the anti-mCRP mAbs was used. The upper row outlines untreated SK-OV3 cells. The middle row represents SK-OV3 cells transfected with Ctrl-siRNA and the lower row depicts SK-OV3 cells treated with triple siRNAs (CD46, CD55 and CD59). The bar values and standard deviation were calculated from triplicates.

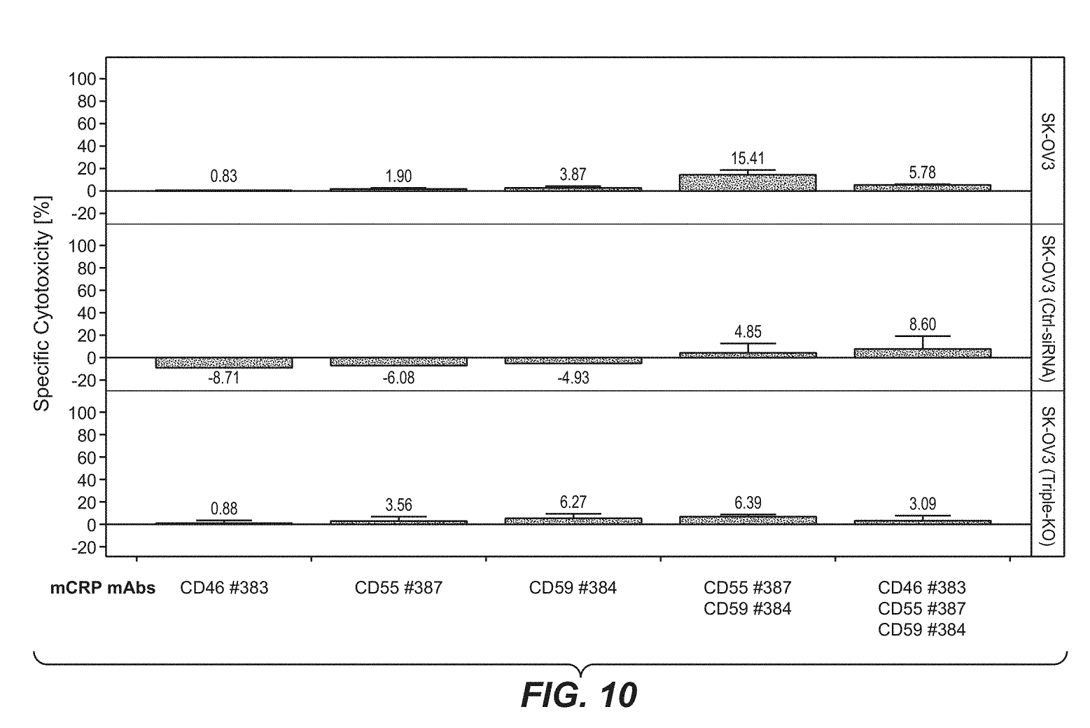

FIG. 10: Results of NHS CDC-assay without Per+Tra. The antibodies Per+Tra were not used in this experiment. The human complement (NHS) was used in a 1/30 dilution. The tenfold concentration of the anti-mCRP mAbs was used. The upper row outlines untreated SK-OV3 cells. The middle row represents SK-OV3 cells transfected with Ctrl-siRNA and the lower row depicts SK-OV3 cells treated with triple siRNAs (CD46, CD55 and CD59). The bar values and standard deviation were calculated from triplicates.

### DETAILED DESCRIPTION OF EMBODIMENTS OF THE INVENTION

### I. DEFINITIONS

The term “C1q binding” denotes the binding of C1q to an antibody bound to its antigen. The binding of the antibody to its antigen is without limitation in vivo and in vitro within the methods and assays as reported herein.

In one embodiment C1q binding is determined in a method comprising i) coating a multi-well plate (e.g. a 96-well ELISA plate) overnight at 4° C. with antibody in PBS at a concentration ranging from 0.007 to 25.0 mg/mL, ii) washing the plates, iii) blocking remaining reactive surface residues with 0.5× PBS/0.025% Tween 20/0.1% gelatin, iv) incubating the multi-well plates for one hour at 37° C. with a) 3% pooled human serum, b) rabbit anti-human C1q, and c) swine anti-rabbit IgG antibody conjugated to HRP, comprising in-between washing, v) incubating for about 30 min with 1 mg/mL 2,2′-azino-bis 3-ethylbenzothiazoline-6-sulfonic acid, vi) adding 100 μL 2% oxalic acid, and vii) measuring the absorbance at 405 nm in a microplate reader.

C1q binding of an antibody denotes herein a multivalent interaction resulting in high avidity binding.

The term “complement activation” denotes the initiation of the classical complement pathway. This initiation results from the binding of complement component C1q to the antibody-antigen complex. C1q is the first protein in the classical complement cascade. It is involved in a series of reactions that result in the formation of an active C3 convertase, which cleaves complement component C3 into C3b and C3a. C3b binds to membrane C5 resulting in so called C5b which triggers the late events of complement activation (assembly of C5b, C6, C7, C8 and C9 into the membrane attack complex (MAC)). Finally the complement cascade results in the formation of pores in the cell wall causing cell lysis (aka complement dependent cytotoxicity, CDC).

The term “complement-dependent cytotoxicity (CDC)” denotes the process of antibody-mediated complement activation resulting in the lysis of a cell according to the mechanism outlined above upon binding of the antibody to its antigen located on that cell. CDC can be determined in vitro using specific CDC assay. In the art normal human serum is used as a complement source.

The term “complement-dependent cellular cytotoxicity (CDCC)” denotes the process of cell killing mediated by cells expressing complement receptors that recognize complement 3 (C3) cleavage products (located on target cells and resulting from antibody-mediated complement activation).

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (k_{d}). Affinity can be measured by common methods known in the art, including those described herein. Specific illustrative and exemplary embodiments for measuring binding affinity are described in the following.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity and can elicit CDC.

“Effector functions” refer to those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, Md. (1991), NIH Publication 91-3242.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain which are hypervariable in sequence (“complementarity determining regions” or “CDRs”) and form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs herein include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, Md. (1991), NIH Publication 91-3242);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The murine monoclonal antibody 4D5 is targeting HER2 specifically in HER2 overexpressing cancer cells, while having no effect on cells expressing physiological levels of HER2. The humanized (4D5) monoclonal antibody (hu4D5) is commercially known as the drug Herceptin® (trastuzumab, rhuMab HER2, U.S. Pat. No. 5,821,337), which gained FDA marketing approval in late 1998.

Pertuzumab (rhuMab 2C4, U.S. Pat. No. 7,862,817) is a humanized monoclonal antibody, which is designed specifically to prevent the HER2 receptor from pairing (dimerising) with other HER receptors (EGFR/HER1, HER3 and HER4) on the surface of cells, a process that is believed to play a role in tumor growth and survival. Pertuzumab is approved in combination with trastuzumab and docetaxel in adult patients with HER2-positive metastatic or locally recurrent non-resectable breast cancer and gained FDA approval for neoadjuvant breast cancer treatment in September 2013.

Pertuzumab binds to domain II of HER2, essential for dimerization, while trastuzumab binds to extracellular domain IV of HER2.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewing's sarcoma, including refractory versions of any of the above cancers, or a combination of one or more of the above cancers. In one embodiment the cancer is a carcinoma.

The term “antigen-binding site” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

Antibody specificity refers to selective recognition of the antibody for a particular epitope of an antigen. Natural antibodies, for example, are monospecific. The term “monospecific” antibody as used herein denotes an antibody that has one or more binding sites each of which bind to the same epitope of the same antigen.

“Bispecific antibodies” are antibodies which have two different antigen-binding specificities. The term “bispecific” antibody as used herein denotes an antibody that has at least two binding sites each of which bind to different epitopes.

The term “valent” as used within the current application denotes the presence of a specified number of binding sites in an antibody molecule. As such, the terms “bivalent”, “tetravalent”, and “hexavalent” denote the presence of two binding sites, four binding sites, and six binding sites, respectively, in an antibody molecule. The bispecific antibodies according to the invention are at least “bivalent” and may be “trivalent” or “multivalent” (e.g. “tetravalent” or “hexavalent”).

As used herein, the term “binding” or “specifically binding” refers to the binding of the antibody to an epitope of the antigen in an in-vitro assay, preferably in a surface plasmon resonance assay (SPR, BIAcore, GE-Healthcare Uppsala, Sweden). The affinity of the binding is defined by the terms kₐ (rate constant for the association of the antibody from the antibody/antigen complex), k_{d} (dissociation constant), and K_{D} (k_{d}/kₐ). Binding or specifically binding means a binding affinity (K_{D}) of 10⁻⁷ mol/L or less.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The term “CD46” denotes a complement regulatory protein (cluster of differentiation 46). This protein is a type membrane protein and has a function in the regulation of the complement system. The encoded protein has cofactor activity for inactivation of complement components C3b and C4b by serum factor I, which protects the host cell from damage by complement.

The term “CD55” denotes a complement decay-accelerating factor (cluster of differentiation 55). It interacts with factor C4b and C3b fragments in the complement cascade. Its interaction with cell-associated C4b and C3b polypeptides interferes with their ability to catalyze the conversion of C2 and factor B to enzymatically active C2a and Bb and thereby prevents the formation of C4b2a and C3bBb, the amplification convertases of the complement cascade (see UniProtKB—P08174 (DAF_HUMAN); Ward, T., et al., EMBO J. 13 (1994) 5070-5074).

The term “CD59” denotes an inhibitor of the complement membrane attack complex (MAC) action (cluster of differentiation 59). It acts by binding to the C8 and/or C9 complements of the assembling MAC, thereby preventing incorporation of the multiple copies of C9 required for complete formation of the osmolytic pore. This inhibitor appears to be species-specific. Involved in signal transduction for T-cell activation complexed to a protein tyrosine kinase. (see UniProtKB—P13987 (CD59_HUMAN)).

### II. METHODS AS REPORTED HEREIN

Carcinomas are of epithelial origin and the cells often upregulate the mCRPs (especially CD46, CD55 and CD59) as immune escape mechanism evading the CDC pressure in vivo. In some cases carcinoma-cell surface antigen binding antibodies cannot elicit CDC due to the effect/presence of the mCRPs. In the past this has been addressed in carcinoma cells using tedious, complicated and instable approaches, such as e.g. siRNA down-regulation of the mCRPs. Herein is reported an improved, i.e. among other things more robust and high-throughput compatible, assay for the analysis of the CDC capacity of carcinoma-cell surface antigen binding antibodies. Thereby a more robust assay for the analysis of the CDC capacity of carcinoma-cell binding antibodies using NHS is provided.

It has been found that for determining complement dependent cytotoxicity of a composition that comprises molecules that on the one hand specifically bind to one or more cell surface antigens and that on the other hand comprise an Fc-region polypeptide of human origin, e.g. a combination of two or more human or humanized antibodies or a human or humanized bispecific antibody, a mixture of anti-mCRP antibodies, i.e. a mixture of an anti-CD46 antibody, an anti-CD55 antibody and an anti-CD59 antibody, has to be used.

It has been found that combinations of antibodies blocking human mCRPs are able to allow the induction of CDC on human carcinoma cells with a human IgG1 antibody pair in the presence of normal human serum. This results in an acceleration of the CDC assay by several days and reduces the required amount of manipulatory steps (e.g. especially reducing the pipetting steps).

It has been found that the use of a combination of three mCRP-blocking antibodies (a mixture of an anti-CD46, an anti-CD55, and an anti-CD59 antibody) results in a similar read-out as the reference labor-intensive siRNA approach.

It has further been found that due to the species specificity of the NHS (normal human serum) and the human mCRPs the addition of further non-human antibodies (e.g. with murine Fc-regions) do not induce/increase CDC in the absence of (i.e. without) the respective CDC-inducing human antibody pair (being present).

Using a mixture of anti-mCRP antibodies for blocking of mCRPs instead of using siRNAs has several advantages, such as a reduction of the time required for mCRP blocking (reduction from 3-6 days to a few minutes), and reduction of the manipulative steps (the pipetting effort can be reduced to a minimum).

The current approach counteracts the up-regulation of mCRPs in carcinoma cells (of epithelial origin) as immune escape mechanism for evading the CDC pressure in the body by the addition of a combination of anti-mCRP antibodies. In contrast to epithelial cancer cells this is not a major response in lymphoid tumor cells. The upregulation of mCRPs as an immune escape mechanism is much more pronounced in most of the cancer cells than in contrast to lymphomas or melanomas (see, e.g., Fishelson, Z., et al., Mol. Immunol. 40 (2003) 109-123).

Hellström, I., et al. (J. Immunol. 127 (1981) 157-160) disclosed that the assessing of the CDC capability of monoclonal antibodies is not possible when using carcinoma cells (epithelial origin) in contrast to other target cell lines of other tissue types. The monoclonal antibody pairs 96.5 and 118.1 are only able to kill tumor cells with complement using tumor cells of non-epithelial origin like melanomas or sarcomas, whereas carcinomas of epithelial origin could not be killed in this setting by complement using antibody pairs. This setting is without inhibition of mCRPs.

Additionally, the mCRP repertoire on lymphoid cells compared to epithelial cancer cells is different and therefore less active towards antibodies with human Fc-region.

Herein is reported a method to determine the CDC-activity of antibody combinations or of bispecific antibodies. The method is especially useful in cases in which the incubation with human serum and human cancer cells using other assay set-ups does not provide for a reliable result.

One aspect as reported herein is a method for determining complement dependent cytotoxicity of a composition comprising i) a first binding site that specifically binds to a first epitope on a first antigen, which is conjugated to a first Fc-region polypeptide of human origin, and ii) a second binding site that specifically binds to a second epitope on a second antigen, which is conjugated to a second Fc-region polypeptide of human origin, wherein the method comprises the following steps:
a) incubating a cell expressing the first antigen and the second antigen with the composition and a mixture of anti-mCRP antibodies,
b) adding normal human serum or rabbit complement to the mixture of a), and
c) determining cell lysis and thereby determining complement dependent cytotoxicity of the composition.

One aspect as reported herein is a method for selecting a composition comprising i) a first binding site that specifically binds to a first epitope on a first antigen, which is conjugated to a first Fc-region polypeptide of human origin, and ii) a second binding site that specifically binds to a second epitope on a second antigen, which is conjugated to a second Fc-region polypeptide of human origin that has CDC-activity, wherein the method comprises the following steps:
a) incubating individually a cell expressing the first antigen and the second antigen with two or more compositions and a mixture of anti-mCRP antibodies,
b) adding normal human serum or rabbit complement to the mixture of a),
c) determining cell lysis and thereby determining complement dependent cytotoxicity of the composition, and
d) selecting based on the result of step c) a composition that has CDC-activity.

One aspect as reported herein is a method for determining complement dependent cytotoxicity of an antibody comprising i) at least a first binding site that specifically binds to a first epitope on a first antigen, ii) optionally a second binding site that specifically binds to a second epitope on a second antigen, wherein the method comprises the following steps:
a) incubating a cell expressing at least the first antigen and optionally the second antigen with the antibody and a mixture of anti-mCRP antibodies,
b) adding normal human serum or rabbit complement to the mixture of a), and
c) determining cell lysis and thereby determining complement dependent cytotoxicity of the antibody.

It has been found that monospecific antibodies do not work in the assay as reported herein.

It has surprisingly been found that the combination of i) human cancer cells, ii) a (bispecific) human or humanized antibody or a composition comprising such antibodies, iii) a mixture of anti-mCRP antibodies, and iv) normal human serum or rabbit complement results in a functional assay.

In one embodiment the cell expresses the first antigen and the second antigen.

In one embodiment the first antigen and the second antigen are cell surface antigens.

The cell expressing the cell surface antigens can be any cell. In one embodiment the cell is a cancer cell. In one embodiment the cancer cell is a carcinoma cell.

Complement dependent cytotoxicity should be determined one or two hours after the addition of complement. Thus, in one embodiment cell lysis is determined between 0.5 hours and 3 hours after the addition of complement, i.e. of Baby Rabbit complement. In one embodiment cell lysis is determined between 1 hour and 2 hours after the addition of complement.

Cell lysis can be determined with any suitable method, such as e.g. LDH release or cell viability determination. Thus, in one embodiment cell lysis is determined by determining LDH release or cell viability.

The method as reported herein can be used for the selection of antibody combinations which do not cross-compete with each other for binding but to exert CDC in combination (not alone).

One aspect as reported herein is a method for determining complement dependent cytotoxicity of a composition

wherein the composition comprises
i) a first binding site that specifically binds to a first epitope on a first antigen, which is conjugated to a first Fc-region polypeptide of human origin, and
ii) a second binding site that specifically binds to a second epitope on the first antigen or on a second antigen, which is conjugated to a second Fc-region polypeptide of human origin,

wherein the method comprises the following steps:
a) incubating a human cell expressing the first antigen or the first antigen and the second antigen with the composition and a mixture of anti-mCRP antibodies,
b) adding normal human serum or rabbit complement to the mixture of a), and
c) determining cell lysis and thereby determining complement dependent cytotoxicity of the composition.

One aspect as reported herein is a method for determining complement dependent cytotoxicity of a combination of two monospecific antibodies or of a bispecific antibody

wherein
i) the first monospecific antibody specifically binds to a first epitope on a first antigen, and the second monospecific antibody specifically binds to a second epitope on the first antigen or on a second antigen, or
ii) the bispecific antibody comprises a first binding site that specifically binds to a first epitope on a first antigen, and a second binding site that specifically binds to a second epitope on the first antigen or on a second antigen

wherein the method comprises the following steps:
a) incubating a human carcinoma cell of epithelial origin expressing the first antigen or the first antigen and the second antigen with the combination of the two monospecific antibodies or with the bispecific antibody and a mixture of anti-mCRP antibodies,
b) adding normal human serum or rabbit complement to the mixture of a), and
c) determining cell lysis and thereby determining complement dependent cytotoxicity of the combination of two monospecific antibodies or of the bispecific antibody.

### Humanized Antibodies

Typically, a non-human antibody that is intended to be used as therapeutic is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of or a full length human constant region. In some embodiments, some FR residues in a humanized antibody are substituted with corresponding residues from a non-human antibody (e.g. the antibody from which the HVR residues are derived), e.g. to restore or improve antibody specificity or affinity.

Humanized antibodies and methods of making them are reviewed, e.g., in Almagro, J. C. and Fransson, J., Front. Biosci. 13 (2008) 1619-1633, and are further described, e.g., in Riechmann, I. et al., Nature 332 (1988) 323-329; Queen, C. et al., Proc. Natl. Acad. Sci. USA 86 (1989) 10029-10033; U.S. Pat. Nos. 5,821,337, 7,527,791, 6,982,321, and 7,087,409; Kashmiri, S. V., et al., Methods 36 (2005) 25-34 (describing specificity determining region (SDR) grafting); Padlan, E. A., Mol. Immunol. 28 (1991) 489-498 (describing “resurfacing”); Dall'Acqua, W. F., et al., Methods 36 (2005) 43-60 (describing “FR shuffling”); Osbourn, J., et al., Methods 36 (2005) 61-68; and Klimka, A., et al., Br. J. Cancer 83 (2000) 252-260 (describing the “guided selection” approach to FR shuffling).

Human framework regions that may be used for humanization include but are not limited to: framework regions selected using the “best-fit” method (see, e.g., Sims, M. J., et al., J. Immunol. 151 (1993) 2296-2308; framework regions derived from the consensus sequence of human antibodies of a particular subgroup of light or heavy chain variable regions (see, e.g., Carter, P., et al., Proc. Natl. Acad. Sci. USA 89 (1992) 4285-4289; and Presta, L. G., et al., J. Immunol. 151 (1993) 2623-2632); human mature (somatically mutated) framework regions or human germline framework regions (see, e.g., Almagro, J. C. and Fransson, J., Front. Biosci. 13 (2008) 1619-1633); and framework regions derived from screening FR libraries (see, e.g., Baca, M., et al., J. Biol. Chem. 272 (1997) 10678-10684; and Rosok, M. J., et al., J. Biol. Chem. 271 (19969 22611-22618).

### Multispecific Antibodies

In certain embodiments, an antibody used in the method reported herein is a multispecific antibody, e.g. a bispecific antibody. Multispecific antibodies are monoclonal antibodies that have binding specificities for at least two different sites/antigens/epitopes. In certain embodiments, bispecific antibodies may bind to two different epitopes of the same antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (see WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553); using “diabody” technology for making bispecific antibody fragments (see, e.g., Holliger, P., et al., Proc. Natl. Acad. Sci. USA 90 (1993) 6444-6448); using single-chain Fv (sFv) dimers (see, e.g. Gruber, M., et al., J. Immunol. 152 (1994) 5368-5374); and preparing trispecific antibodies as described, e.g., in Tutt, A., et al., J. Immunol. 147 (1991) 60-69).

Engineered antibodies with three or more functional antigen binding sites, including “Octopus antibodies,” are also included herein (see, e.g. US 2006/0025576).

The antibody also includes a “Dual Acting Fab” or “DAF” (see, US 2008/0069820, for example).

The antibody or fragment herein also includes multispecific antibodies described in WO 2009/080251, WO 2009/080252, WO 2009/080253, WO 2009/080254, WO 2010/112193, WO 2010/115589, WO 2010/136172, WO 2010/145792, and WO 2010/145793.

### Recombinant Methods and Compositions

Antibodies may be produced using recombinant methods and compositions, e.g., as described in U.S. Pat. No. 4,816,567. For expression nucleic acids encoding the individual polypeptide chains of the antibody are required. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, the nucleic acid(s) encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523; see also Charlton, K. A., In: Methods in Molecular Biology, Vol. 248, Lo, B.K.C. (ed.), Humana Press, Totowa, N.J. (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (see Gerngross, T. U., Nat. Biotech. 22 (2004) 1409-1414; and Li, H., et al., Nat. Biotech. 24 (2006) 210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (see, e.g., U.S. Pat. Nos. 5,959,177, 6,040,498, 6,420,548, 7,125,978, and 6,417,429 (describing PLANTIBODIES™ technology for producing antibodies in transgenic plants)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR⁻ CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B.K.C. (ed.), Humana Press, Totowa, N.J. (2004), 255-268.

### Pharmaceutical Formulations

Pharmaceutical formulations of antibodies are prepared by mixing such antibodies having the desired degree of purity with one or more optional pharmaceutically acceptable carriers (Remington's Pharmaceutical Sciences, 16th edition, Osol, A. (ed.) (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyl dimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) peptides; proteins, such as serum albumin, gelatin, or immunoglobulins; hydrophilic polymers such as poly(vinylpyrrolidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH20 (HYLENEX®, Baxter International, Inc.). Certain exemplary sHASEGPs and methods of use, including rhuPH20, are described in US 2005/0260186 and US 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Any of the compositions, i.e. antibody combinations or multispecific antibodies, selected with a method provided herein may be used in therapeutic methods.

In one aspect, a composition selected with a method as reported herein for use as a medicament is provided. In certain embodiments, a composition selected with a method as reported herein for use in a method of treatment is provided. In certain embodiments, the invention provides a composition selected with a method as reported herein for use in a method of treating an individual comprising administering to the individual an effective amount of the composition selected with a method as reported herein. In one such embodiment, the method further comprises administering to the individual an effective amount of at least one additional therapeutic agent. An “individual” according to any of the above embodiments is preferably a human.

In a further aspect, the invention provides for the use of a composition selected with a method as reported herein in the manufacture or preparation of a medicament. In a further embodiment, the composition selected with a method as reported herein is for use in a method of treating a disease comprising administering to an individual having the disease an effective amount of the composition selected with a method as reported herein. In one such embodiment, the method further comprises administering to the individual an effective amount of at least one additional therapeutic agent. An “individual” according to any of the above embodiments may be a human.

In a further aspect, the invention provides a method for treating a disease. In one embodiment, the method comprises administering to an individual having such disease an effective amount of a composition selected with a method as reported herein. In one such embodiment, the method further comprises administering to the individual an effective amount of at least one additional therapeutic agent. An “individual” according to any of the above embodiments may be a human.

In a further aspect, the invention provides pharmaceutical formulations comprising a composition selected with a method as reported herein, e.g., for use in any of the above therapeutic methods. In one embodiment, a pharmaceutical formulation comprises any of the compositions selected with a method as reported herein and a pharmaceutically acceptable carrier. In another embodiment, a pharmaceutical formulation comprises any of the compositions selected with a method as reported herein and at least one additional therapeutic agent.

Compositions selected with a method as reported herein can be used either alone or in combination with other agents in a therapy. For instance, a composition selected with a method as reported herein may be co-administered with at least one additional therapeutic agent.

Such combination therapies noted above encompass combined administration (where two or more therapeutic agents are included in the same or separate formulations), and separate administration, in which case, administration of the composition selected with a method as reported herein can occur prior to, simultaneously, and/or following, administration of the additional therapeutic agent or agents. In one embodiment, administration of the composition selected with a method as reported herein and administration of an additional therapeutic agent occur within about one month, or within about one, two or three weeks, or within about one, two, three, four, five, or six days, of each other.

A composition selected with a method as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Compositions selected with a method as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The composition selected with a method as reported herein need not be, but is optionally formulated with one or more agents currently used to prevent or treat the disorder in question. The effective amount of such other agents depends on the amount of the components present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a composition selected with a method as reported herein (when used alone or in combination with one or more other additional therapeutic agents) will depend on the type of disease to be treated, the type of composition, the severity and course of the disease, whether the composition is administered for preventive or therapeutic purposes, previous therapy, the patient's clinical history and response to the composition, and the discretion of the attending physician. The composition selected with a method as reported herein is suitably administered to the patient at one time or over a series of treatments. Depending on the type and severity of the disease, about 1 μg/kg to 15 mg/kg (e.g. 0.5 mg/kg-10 mg/kg) of composition can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the composition would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

### III. EXAMPLES

The following are examples of methods and compositions of the invention. It is understood that various other embodiments may be practiced, given the general description provided above.

### Materials and Methods

### Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions.

### Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany)

### Reagents

All commercial chemicals, antibodies and kits were used as provided according to the manufacturer's protocol if not stated otherwise.

### Materials

| Chemical/Reagent | Supplier |
|---|---|
| AIM-V Serum Free Medium | Gibco |
| Baby Rabbit Complement | Cedarlane |
| CD45, CD55, CD59, Ctrl-siRNA | Biospring |
| LDH Reaction Mix | Roche Diagnostics GmbH |
| LipofectAmine RNAi MAX | Invitrogen |
| Triton-X 100 | Roche Diagnostics GmbH |

### Cell Lines

| Cell name | Disease |
|---|---|
| SK-OV3 | ovary adenocarcinoma, human |
| MCF7 | breast adenocarcinoma, human |

### Cell Growth Media

| Cell name | Medium composition |
|---|---|
| MCF7 | 90% Eagles MEM + Earles BSS |
| | 10% FCS |
| | 2 mM L-Glutamine |
| | 1 mM NEAA |
| | 1 mM Sodium Pyruvate |
| SK-OV3 | 90% McCoys |
| | 10% FCS |
| | 2 mM L-Glutamine |
| | 1 mM NEAA |
| | 1 mM Sodium Pyruvate |

### Antibodies

| | | | | | Provider, |
|---|---|---|---|---|---|
| Antibody | Antibody | | | | Catalogue |
| Number | against | Host | Label | Isotype | number |
| #362 | CD59, | mouse | PE | mAb, IgG2a | BD Pharmingen, |
| | human | | | | 555764 |
| #363 | CD55, | mouse | APC | mAb, IgG2a | BD Pharmingen, |
| | human | | | | 555696 |
| #364 | CD46, | mouse | FITC | mAb, IgG2a | BD Pharmingen, |
| | human | | | | 555949 |
| #383 | CD46, | mouse | — | mAb, IgG1 | Hycult, |
| | human | | | | HM2103 |
| #384 | CD59, | mouse | — | mAb, IgG2a | antibodies online, |
| | human | | | | ABIN118751 |
| #385 | CD59, | rat | — | mAb, | AbD Serotec, |
| | human | | | IgG2b | MCA715G |
| #386 | CD46, | mouse | — | IgG1 | AbD Serotec, |
| | human | | | | MCA2113 |
| #387 | CD55, | mouse | — | IgG1 | AbD Serotec, |
| | human | | | | MCA914 |

### Recombinantly Produced Antibodies

trastuzumab
light chain:
(SEQ ID NO: 01)
DIQMTQSPSS LSASVGDRVT ITCRASQDVN TAVAWYQQKP
GKAPKLLIYS ASFLYSGVPS RFSGSRSGTD FTLTISSLQP
EDFATYYCQQ HYTTPPTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
heavy chain:
(SEQ ID NO: 02)
EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSREE
MTKNQVSLTC LVKGFYPSDI AVEWESNGQP ENNYKTTPPV
LDSDGSFFLY SKLTVDKSRW QQGNVFSCSV MHEALHNHYT
QKSLSLSPGK
pertuzumab
light chain:
(SEQ ID NO: 03)
DIQMTQSPSS LSASVGDRVT ITCKASQDVS IGVAWYQQKP
GKAPKLLIYS ASYRYTGVPS RFSGSGSGTD FTLTISSLQP
EDFATYYCQQ YYIYPYTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
heavy chain:
(SEQ ID NO: 04)
EVQLVESGGG LVQPGGSLRL SCAASGFTFT DYTMDWVRQA
PGKGLEWVAD VNPNSGGSIY NQRFKGRFTL SVDRSKNTLY
LQMNSLRAED TAVYYCARNL GPSFYFDYWG QGTLVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKKVEPK SCDKTHTCPP CPAPELLGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSRDEL
TKNQVSLTCL VKGFYPSDIA VEWESNGQPE NNYKTTPPVL
DSDGSFFLYS KLTVDKSRWQ QGNVFSCSVM HEALHNHYTQ
KSLSLSPGK
bispecific anti-HER2 antibody, light chain
light chain:
(SEQ ID NO: 05)
DIQMTQSPSS LSASVGDRVT ITCKASQDVS TAVAWYQQKP
GKAPKLLIYS ASFRYTGVPS RFSGSRSGTD FTLTISSLQP
EDFATYYCQQ HYTTPPTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
heavy chain 1 (knob, trastuzumab):
(SEQ ID NO: 06)
EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPCRDE
LTKNQVSLWC LVKGFYPSDI AVEWESNGQP ENNYKTTPPV
LDSDGSFFLY SKLTVDKSRW QQGNVFSCSV MHEALHNHYT
QKSLSLSPGK
heavy chain 2 (hole, pertuzumab):
(SEQ ID NO: 07)
EVQLVESGGG LVQPGGSLRL SCAASGFTFN DYTMDWVRQA
PGKGLEWVAD VNPNSGGSIV NRRFKGRFTL SVDRSKNTLY
LQMNSLRAED TAVYYCARNL GPFFYFDYWG QGTLVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKKVEPK SCDKTHTCPP CPAPELLGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV CTLPPSRDEL
TKNQVSLSCA VKGFYPSDIA VEWESNGQPE NNYKTTPPVL
DSDGSFFLVS KLTVDKSRWQ QGNVFSCSVM HEALHNHYTQ
KSLSLSPGK
bispecific anti-HER2 antibody, CrossMab format:
heavy chain 1:
(SEQ ID NO: 08)
QVQLVQSGAE VKKPGASVKV SCKASGFNIK DTYIHWVRQA
PGQGLEWMGR IYPTNGYTRY AQKFQGRVTM TRDTSISTAY
MELSRLRSDD TAVYYCSRWG GEGFYAMDYW GQGTMVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VCTLPPSRDE
LTKNQVSLSC AVKGFYPSDI AVEWESNGQP ENNYKTTPPV
LDSDGSFFLV SKLTVDKSRW QQGNVFSCSV MHEALHNHYT
QKSLSLSPGK
heavy chain 2:
(SEQ ID NO: 09)
EVQLVESGGG LVQPGGSLRL SCAASGFTFT DYTMDWVRQA
PGKGLEWVAD VNPNSGGSIY NQRFKGRFTL SVDRSKNTLY
LQMNSLRAED TAVYYCARNL GPSFYFDYWG QGTLVTVSSA
SVAAPSVFIF PPSDEQLKSG TASVVCLLNN FYPREAKVQW
KVDNALQSGN SQESVTEQDS KDSTYSLSST LTLSKADYEK
HKVYACEVTH QGLSSPVTKS FNRGECDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPC
RDELTKNQVS LWCLVKGFYP SDIAVEWESN GQPENNYKTT
PPVLDSDGSF FLYSKLTVDK SRWQQGNVFS CSVMHEALHN
HYTQKSLSLS PGK
light chain 1:
(SEQ ID NO: 10)
DIQLTQPPSV SVAPGQTARI TCGASQDVST AVAWYQQKPG
QAPVLVVYSA SFLYSGIPSR FSGSRSGTDF TLTISRVEAG
DEADYYCQQH YTTPPTFGTG TKVTVLRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC
light chain 2:
(SEQ ID NO: 11)
DIQMTQSPSS LSASVGDRVT ITCKASQDVS IGVAWYQQKP
GKAPKLLIYS ASYRYTGVPS RFSGSGSGTD FTLTISSLQP
EDFATYYCQQ YYIYPYTFGQ GTKVEIKSSA STKGPSVFPL
APSSKSTSGG TAALGCLVKD YFPEPVTVSW NSGALTSGVH
TFPAVLQSSG LYSLSSVVTV PSSSLGTQTY ICNVNHKPSN
TKVDKKVEPK SC

### Expression

### a) Construction of the Expression Plasmids

The following expression vector was used for the construction of all heavy and light chain encoding expression plasmids. The vector is composed of the following elements:
a hygromycin resistance gene as a selection marker,
an origin of replication, oriP, of Epstein-Barr virus (EBV),
an origin of replication from the vector pUC18 which allows replication of this plasmid in E. coli,
a beta-lactamase gene which confers ampicillin resistance in E. coli,
the immediate early enhancer and promoter from the human cytomegalovirus (HCMV),
the human immunoglobulin polyadenylation (“poly A”) signal sequence.

The immunoglobulin genes comprising the heavy or light chain were prepared by gene synthesis and cloned into pGA18 (ampR) plasmids as described above. Variable heavy chain constructs were constructed by directional cloning using unique restriction sites. Variable light chain constructs were ordered as gene synthesis comprising VL and CL and constructed by directional cloning using unique restriction sites. The final expression vectors were transformed into E. coli cells, expression plasmid DNA was isolated (Miniprep) and subjected to restriction enzyme analysis and DNA sequencing. Correct clones were grown in 150 ml LB-Amp medium, again plasmid DNA was isolated (Maxiprep) and sequence integrity confirmed by DNA sequencing.

### b) Transient Expression of Immunoglobulin Variants in HEK293 Cells

Recombinant immunoglobulins were expressed by transient transfection of human embryonic kidney 293-F cells using the FreeStyle™ 293 Expression System according to the manufacturer's instruction (Invitrogen, USA). For small scale test expressions 30 mL of 0.5×10⁶ HEK293F cells/mL were seeded one day prior to transfection. The next day, plasmid DNA (1 μg DNA per mL culture volume) was mixed with 1.2 mL Opti-MEM® I Reduced Serum Medium (Invitrogen, Carlsbad, Calif., USA) followed by addition of 40 μL of 293Fectin™ Transfection Reagent (Invitrogen, Carlsbad, Calif., USA). The mixture was incubated for 15 min. at room temperature and added drop wise to the cells. One day post-transfection each flask was fed with 300 μL L-glutamine (200 mM, Sigma-Aldrich, Steinheim, Germany) and 600 μL of a feed containing amino acids, sugar, trace elements, FreeStyle medium without RPMI. Three days post-transfection cell concentration, viability and glucose concentration in the medium were determined using an automated cell viability analyzer (Vi-CELL™ XR, Beckman Coulter, Fullerton, Calif., USA) and a glucose meter (Accu-CHEK® Sensor comfort, Roche Diagnostics GmbH, Mannheim, Germany). In addition each flask was fed with 300 μL of L-glutamine, 300 μL non-essential amino acids solution (PAN™ Biotech, Aidenbach, Germany), 300 μL sodium pyruvate (100 mM, Gibco, Invitrogen), 1.2 ml feed and ad 5 g/L glucose (D-(+)-glucose solution 45%, Sigma). Finally, six days post-transfection antibodies were harvested by centrifugation at 3500 rpm in a X3R Multifuge (Heraeus, Buckinghamshire, England) for 15 min. at ambient temperature, the supernatant was sterile filtered through a Steriflip filter unit (0.22 μm Millipore Express PLUS PES membrane, Millipore, Bedford, Mass.) and stored at −20° C. until further use. Large scale transfections up to 5 L were scaled linearly.

### c) Purification

Bispecific antibodies were purified from cell culture supernatants by affinity chromatography using Protein A-Sepharose™ (GE Healthcare, Sweden) and Superdex200 size exclusion chromatography. Briefly, sterile filtered cell culture supernatants were applied on a HiTrap Protein A HP (5 mL) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. Antibody and antibody variants were eluted with 0.1 M citrate buffer, pH 2.8, and the protein containing fractions were neutralized with 0.1 mL 1 M Tris, pH 8.5. Eluted protein fractions were pooled, concentrated with an Amicon Ultra centrifugal filter device (MWCO: 30 K, Millipore) to a volume of 3 mL and loaded on a Superdex200 HiLoad 120 mL 16/60 gel filtration column (GE Healthcare, Sweden) equilibrated with 20 mM histidine, 140 mM NaCl, pH 6.0. Fractions containing purified bispecific and control antibodies with less than 5% high molecular weight aggregates were pooled and stored as 1.0 mg/mL aliquots at −80° C.

### d) Protein Quantification

Proteins were quantified by affinity chromatography using the automated Ultimate 3000 system (Dionex, Idstein, Germany) with a pre-packed Poros® A Protein A column (Applied Biosystems, Foster City, Calif., USA). All samples were loaded in buffer A (0.2 M Na₂HPO₄.[2 H₂O], pH 7.4) and eluted in buffer B (0.1 M citric acid, 0.2 M NaCl, pH 2.5). In order to determine the protein concentration an extinction coefficient of 1.62 was used for all samples.

### e) Analysis of Purified Proteins

The protein concentration of purified protein samples was determined by measuring the optical density (OD) at 280 nm, using the molar extinction coefficient calculated on the basis of the amino acid sequence. Purity and molecular weight of bispecific and control antibodies were analyzed by SDS-PAGE in the presence and absence of a reducing agent (5 mM 1,4-dithiotreitol) and staining with Coomassie brilliant blue. The NuPAGE® Pre-Cast gel system (Invitrogen, USA) was used according to the manufacturer's instruction (4-20% Tris-Glycine gels). The aggregate content of bispecific and control antibody samples was analyzed by high-performance SEC using a Superdex 200 analytical size-exclusion column (GE Healthcare, Sweden) in 200 mM KH₂PO₄, 250 mM KCl, pH 7.0 running buffer at 25° C. 25 μg protein were injected on the column at a flow rate of 0.5 mL/min and eluted isocratic over 50 minutes. Integrity of the amino acid backbone of reduced bispecific antibody light and heavy chains was verified by NanoElectrospray Q-TOF mass spectrometry after removal of N-glycans by enzymatic treatment with Peptide-N-Glycosidase F (Roche Molecular Biochemicals).

### f) Analytical HPLC

Antibodies were analyzed using a Agilent HPLC 1100 (Agilent Technologies, Palo Alto, Calif., USA) with a TSK-GEL G3000SW gel filtration column (7.5 mm ID×30 cm, Tosohaas Corp., Montgomeryville, Pa., USA). 18 μL of the eluted proteins were loaded onto the column in Buffer A (0.05 M K₂HPO₄/KH₂PO₄ in 300 mM NaCl, pH 7.5) and separated based on size.

### Example 1

### Assay using Different Complement Sources

Alamar Blue Assay with Guinea Pig Complement (GPC)

CHO-K1 Nxre19 cells (IL15R transfected CHO-K1) were seeded at 20,000 cells/well on 96-well flat bottom cell culture plates (NUNC, 100 μL/well) in DMEM/F12 medium supplemented with GlutaMax (Gibco, Cat. No. 31331-028). Twenty-five microliter of IL15-Fc fusion polypeptide (6-fold end-concentration) were added and incubated for one hour. Thereafter 25 μL of Guinea Pig complement (Sigma Aldrich, Cat. No. S1639) was added and incubated for 3.5 hours. Afterwards 50 μL of Alamar Blue (Promega) was added and incubated overnight at 37° C./5% CO₂. The plates were measured at a wavelength of 550 nm (excitation) and 595 nm (emission).

| | signal | |
|---|---|---|
| sample | [AU] | variation coefficient |
| cells only | 16290 | 240 |
| 2.5 μg/mL IL15-Fc-fusion without GPC | 16408 | 161 |
| complement only without IL15-Fc-fusion | 4893 | 207 |
| 2.5 μg/ml IL15-Fc-fusion with GPC | 4410 | 360 |
| 1.25 μg/ml IL15-Fc-fusion with GPC | 4104 | 163 |
| 0.625 μg/ml IL15-Fc-fusion with GPC | 4397 | 299 |
| 0.3125 μg/ml IL15-Fc-fusion with GPC | 4070 | 104 |
| 0.156 μg/ml IL15-Fc-fusion with GPC | 3944 | 198 |
| 0.078 μg/ml IL15-Fc-fusion with GPC | 3817 | 117 |
| 0.039 μg/ml IL15-Fc-fusion with GPC | 4047 | 29 |
| 0.020 μg/ml IL15-Fc-fusion with GPC | 4432 | 293 |
| 0.010 μg/ml IL15-Fc-fusion with GPC | 4381 | 293 |
| 0.005 μg/ml IL15-Fc-fusion with GPC | 4092 | 89 |

From the data it can be seen that Guinea pig complement is toxic at all dilutions even in the absence of Fc-region.

LDH Assay with Human Complement (HUC)

CHO-K1 Nxre19 cells (IL15R transfected CHO-K1) were seeded at 10,000 cells/well on 96-well flat bottom cell culture plates (NUNC, 100 μL/well) and cultivated overnight in DMEM/F12 medium supplemented with GlutaMax (Gibco, Cat. No. 31331-028). IL15-Fc fusion polypeptide was added (25 μL/well in 5-fold end-concentration) and incubated for one hour. Growth medium was removed and cells were washed once with serum-free medium. Thereafter 190 μL/well serum-free medium and 10 μL of Human complement (Sigma Aldrich, Cat. No. S1764, c=1 mg/mL) was added. After four hours plates were centrifuged at 200 g and 100 μL/well were transferred to another 96-well flat bottom plate. Thereafter 100 μL of LDH reaction mix (Cytotoxicity Detection Kit, Roche Diagnostics GmbH, Mannheim, Germany) were added. After an incubation of 20 min. at 37° C. the optical density (OD) was measured at 492/690 nm on a Tecan Sunrise reader.

| | signal [OD] | |
|---|---|---|
| sample | experiment 1 | experiment 2 |
| 1000 ng/ml IL15-Fc-fusion with HUC | 29.1 | 42.6 |
| 333.3 ng/ml IL15-Fc-fusion with HUC | 32.9 | 42.8 |
| 111.1 ng/ml IL15-Fc-fusion with HUC | 34.0 | 43.1 |
| 37.04 ng/ml IL15-Fc-fusion with HUC | 35.5 | 39.6 |
| 12.35 ng/ml IL15-Fc-fusion with HUC | 37.0 | 39.0 |
| 4.12 ng/ml IL15-Fc-fusion with HUC | 38.4 | 40.7 |
| 1.37 ng/ml IL15-Fc-fusion with HUC | 37.2 | 42.2 |
| 0.46 ng/ml IL15-Fc-fusion with HUC | 29.9 | 32.7 |
| 0 ng/ml IL15-Fc-fusion with HUC | 27.7 | 27.7 |

From the data above it can be seen that Human complement does not exert a dose dependent complement dependent toxicity.

LDH Assay with Baby Rabbit Complement (BRC)

CHO-K1 Nxre19 cells (IL15R transfected CHO-K1) were seeded at 10,000 cells/well on 96-well flat bottom cell culture plates (NUNC, 100 μL/well) and cultivated overnight in DMEM/F12 medium supplemented with GlutaMax (Gibco, Cat. No. 31331-028). IL15-Fc fusion polypeptide was added (25 μL/well in 5-fold end-concentration) and incubated for one hour. Thereafter, one vial of Baby Rabbit complement (Cedarlane, Cat. No. CL3441) was reconstituted with 1 mL of Aqua bidest. The complement solution was diluted with medium and 25 μL added to the wells. After four hours the plates were centrifuged at 200 g and 100 μL/well were transferred to another 96-well flat bottom plate. Thereafter 100 μL of LDH reaction mix (Cytotoxicity Detection Kit, Roche Diagnostic GmbH, Mannheim, Germany) was added. After an incubation time of 20 min. at 37° C. optical density (OD) was measured at 492/690 nm on a Tecan Sunrise reader.

signal [OD]

| sample | BRC 1/40 | BRC 1/30 |
|---|---|---|
| 9000 ng/ml IL15-Fc-fusion with BRC | 11.3 | 12.3 |
| 3000 ng/ml IL15-Fc-fusion with BRC | 12.3 | 17.0 |
| 1000 ng/ml IL15-Fc-fusion with BRC | 10.2 | 13.6 |
| 333.3 ng/ml IL15-Fc-fusion with BRC | 7.8 | 12.2 |
| 111.1 ng/ml IL15-Fc-fusion with BRC | 8.3 | 13.0 |
| 37.04 ng/ml IL15-Fc-fusion with BRC | 14.9 | 19.7 |
| 12.35 ng/ml IL15-Fc-fusion with BRC | 43.2 | 53.0 |
| 4.12 ng/ml IL15-Fc-fusion with BRC | 41.5 | 63.8 |
| 0 ng/ml IL15-Fc-fusion with BRC | 42.4 | 48.4 |

It can be seen that BRC has a low background toxicity and shows dose dependent complement toxicity.

### Example 2

### C1q Binding of Anti-HER2 Antibodies on BT-474 Cells

About 3×10⁵ BT-474 cells were incubated with 10 μg/mL of indicated antibody on ice in RPMI 1640 supplemented with 10% FCS. After 30 min. incubation on ice 10 μg/mL C1q (Sigma Aldrich, Cat. No. C1740) was added. The incubation was continued thereafter for an additionally 20 min. on ice. After washing the cells were resuspended in 200 μL medium and counterstained with a PE-labeled anti-C1q antibody (Cedarlane, Cat. No. CL7611PE-SP). After an incubation time of 30 min. on ice cells were washed twice and analyzed on a FACS Canto II.

| | PE-signal |
|---|---|
| antibody/antibodies | (geomean) |
| trastuzumab | 282 |
| pertuzumab | 344 |
| combination of trastuzumab and pertuzumab | 2157 |
| bispecific anti-HER2 antibody, common light chain | 1439 |
| bispecific anti-HER2 antibody, common light chain, | 1036 |
| glycoengineered | |
| bispecific anti-HER2 antibody, CrossMab format | 489 |

This C1q assay illustrates the binding of recombinant complement factor C1q to different antibodies on BT-474 cells.

### Example 3

### Proliferation Inhibition of Anti-HER2 Antibodies on BT-474 Cells

Ten thousand (1×10⁴) BT-474 cells/well were cultured in RPMI 1640 medium supplemented with 10% FCS in a 96-well flat bottom plate. After 24 hours growth medium was removed and titrated amounts of indicated antibodies were added (premixed in culture medium; 200 nM, 66.7 nM, 22.2 nM, 7.4 nM, 2.5 nM, 0.8 nM, 0.3 nM, 0.1 nM) to a final volume of 100 μL. To determine the number of viable cells in culture, a CellTiterGlo Luminescent Cell Viability Assay according to the manufacturer's instructions was performed (quantifying ATP levels as an indicator of metabolically active cells). Thus, after six days of culture 100 μL CellTiterGlo Reaction Mix (Promega, Cat. No. G7571) was added to the cells and incubated for 2 min. with shaking. Thereafter 75 μL of the lysate was transferred to a separate 96-well flat bottom plate (Costar, Cat. No. 3917). After an additional mixing luminescence was assed according to the manufacturer's instructions using a Tecan Infinite Reader and the respective IC₅₀ value was calculated.

| antibody/antibodies | IC₅₀ [nM] |
|---|---|
| combination of trastuzumab and pertuzumab | 6.20 |
| bispecific anti-HER2 antibody, common light chain | 3.31 |
| bispecific anti-HER2 antibody, common light chain, | 3.93 |
| glycoengineered | |
| bispecific anti-HER2 antibody, CrossMab format | 4.75 |

In the proliferation assay it was shown that the antibodies inhibited proliferation of BT-474.

### Example 4

### CDC Activation by Anti-HER2 Antibodies on BT-474 Cells, SK-Br3 Cells and SK-OV-3 Cells

Ten thousand cells/well (BT-474, SK-Br3 or SK-OV-3 cells) were seeded in a 96-well plate and incubated for 20 hours at 37° C./5% CO₂. Thereafter the medium was removed, the cells were washed once with 100 μL AIM-V medium (Gibco, Cat. No. 0870112 DK). Fifty microliter AIM-V medium were placed in each well. Thereafter 50 μL antibody solution (in 3-fold end-concentration) were added and incubated for 30 min. at 37° C./5% CO₂. Fifty microliter of Baby Rabbit complement (Cedarlane, Cat. No. CL3441, batch no. 6312) 1:10 diluted in AIM-V medium was added and the incubation was continued for 2 hours. Thereafter, 50 μL of the supernatant was transferred and mixed with 50 μL LDH Reaction Mix (Roche Diagnostics GmbH, Mannheim, Germany). After a further incubation of 15 min. at 37° C. extinction (Ex.) was determined at 490/620 nm on a Tecan Sunrise Reader. The specific antibody dependent toxicity (mean+/−SD of n=4) was calculated as follows: % antibody dependent toxicity=(Ex. sample−Ex. spontaneous lysis/Ex. maximal lysis-spontaneous lysis)×100. The results are shown in FIG. 1.

BT474, SkBr3 and SK-OV-3 cells were incubated with trastuzumab, pertuzumab, or a combination thereof (total antibody concentration 10 μg/mL or 1 μg/mL), followed by a two hour incubation with Baby Rabbit complement. Human IgG1 with kappa light chain was used as isotype control. Readout of cell lysis (LDH release) was performed on a Tecan sunrise reader using the LDH Cytotoxicity kit (Roche Diagnostics GmbH, Mannheim, Germany, Cat. No. 11644793001). Specific lysis is given as the signal in relation to 3% Triton-X treated cells (maximum lysis). Experiment was performed in quintuplicates.

| | | specific lysis [%] | | |
|---|---|---|---|---|
| antibody/ | | | | SK-OV-3 |
| antibodies | dosage | BT-474 cells | SkBr3 cells | cells |
| trastuzumab | 10 μg/mL | 12.8 ± 0.9 | −1.1 ± 0.7 | 0.5 ± 1.8 |
| pertuzumab | 10 μg/mL | 7.3 ± 0.6 | −1.4 ± 0.7 | −0.5 ± 1.1 |
| combination | 5 μg/mL + | 179.6 ± 1.3 | 157.2 ± 8.7 | 34.6 ± 9.9 |
| of | 5 μg/mL | | | |
| trastuzumab | | | | |
| and | | | | |
| pertuzumab | | | | |
| human IgG1, | 10 μg/mL | 0.9 ± 0.8 | 5.2 ± 1.5 | −0.7 ± 1.0 |
| kappa | 1 μg/mL | −8.1 ± 0.6 | −7.7 ± 3.1 | 1.3 ± 0.9 |
| trastuzumab | 1 μg/mL | −5.1 ± 0.6 | −2.4 ± 0.2 | 1.5 ± 3.2 |
| pertuzumab | 0.5 μg/mL + | 109.3 ± 5.4 | 64.3 ± 19.8 | 20.9 ± 14.4 |
| combination | 0.5 μg/mL | | | |
| of | | | | |
| trastuzumab | | | | |
| and | | | | |
| pertuzumab | | | | |
| human IgG1, | 1 μg/mL | 10.3 ± 0.6 | 3.6 ± 1.0 | 1.5 ± 1.4 |
| kappa | | | | |

This CDC assay shows the release of LDH as a marker for dying/dead cells upon treatment with different antibodies (formats, combination) in the presence of Baby Rabbit complement.

### Example 5

### Determination of Antibody Ratio for CDC

Ten thousand SK-OV-3 cells per well were seeded into a 96-well flat bottom plate (Thermo Scientific, Nunclon Delta Surface) in 100 μL per well in AIM-V medium (Gibco, Cat. No. 0870112-DK) and were incubated for 20 hours at 37° C. and 5% CO₂. After the incubation period, 50 μL of the antibody-stock solutions containing trastuzumab and pertuzumab at a final concentration of 0.1, 0.5, 1, 5, or 10 μg/mL were added. Human IgG1, kappa light chain (Sigma, Cat. No. I5154-1MG) was used as control. Triton-X (Roche Diagnostics GmbH, Mannheim, Germany, Cat. No. 11332481001) at a final concentration of 1% was added for determination of maximum lysis. After incubation for 30 min. at 37° C. 50 μL Baby Rabbit Complement-stock solution (Cedarlane, Cat. No. CL3441) was added with a final dilution of 1/30. Thereafter the plates were incubated for 2 hours at 37° C. (final volume/well=150 μL). The amount of cell lysis was determined via the LDH activity using the Cytotoxicity Detection Kit (Roche Diagnostics GmbH, Mannheim, Germany, Cat. No. 11644793001). The absorbance was determined at 490 nm and 620 nm with a Tecan Sunrise reader.

As positive control the following samples were used:
medium control: SK-OV-3 cells with AIM-V medium
spontaneous lysis: SK-OV-3 cells with active BRC
maximal lysis: SK-OV-3 cells with 1 Triton-X
isotype control: SK-OV-3 cells with 10 μg/mL human IgG, kappa and BRC
negative control: SK-OV-3 cells with 10 μg/mL antibody/composition and heat inactivated BRC
assay control: SK-OV-3 cells with 10 μg/mL trastuzumab and pertuzumab and active BRC.

An optimal cell killing was observed at trastuzumab/pertuzumab ratios of 0.5:1 to 1:1 as well as at pertuzumab/trastuzumab ratio of 0.5:1 to 1:1. Overall, the assay seemed to be very robust towards the change of the antibody ratio since even a 1:10 ratio did not influence the CDC dramatically.

### Example 6

### CDC-Mediated Killing of BT-474 Cells by Anti-HER2 Antibodies

Ten thousand BT-474 cells/well were seeded on 96-well E-Plates (ACEA Biosciences Inc.) and grown overnight in an Xcelligence device in AIM-V medium. Growth medium was removed and cells were washed once with serum-free AIM-V medium (Gibco). Fifty microliter per well AIM-V medium and 50 μL antibody in AIM-V (3-fold end concentration) were added and incubated for 20 min. Thereafter 50 μL Baby Rabbit complement (Cedarlane) was added and Cell Index (CI; as representative for the viability of the cells) was measured every 5 minutes. Specific CDC was calculated according following formula, whereas CI is the normalized cell index:

$$\% CDC = \frac{CI Complement control - CI sample}{CI Complement control} \times 100$$

At two representative time points (1 hour and 2 hours after starting the reaction, specific lysis (i.e. CDC-induced cell death) was calculated and shown in FIG. 2 and the following Table (mean+/SEM of n=4).

| | specific lysis | |
|---|---|---|
| | [% cell index | |
| | ACEA] | |
| antibody/antibodies | 1 hour | 2 hours |
| trastuzumab | −3.5 ± 0.6 | −6.5 ± 0.8 |
| pertuzumab | −5.3 ± 1.0 | −8.3 ± 2.1 |
| combination of trastuzumab and pertuzumab | 20.9 ± 6.7 | 26.3 ± 7.0 |
| bispecific anti-HER2 antibody, common light | 31.8 ± 3.4 | 38.9 ± 3.7 |
| chain | | |
| bispecific anti-HER2 antibody, common light | 28.8 ± 2.6 | 35.8 ± 2.6 |
| chain, glycoengineered | | |
| bispecific anti-HER2 antibody, CrossMab | 12.9 ± 1.4 | 22.7 ± 1.6 |
| format | | |

This CDC assay illustrates a change in the cell index as a marker for dying/dead cells upon treatment with different antibodies (formats, combination) in the presence of Baby Rabbit complement.

### Example 7

### Attempt to Establish a CDC Assay Based on Complement of Human Origin

SkBr3 cells were sensitized with trastuzumab, pertuzumab, or combination of trastuzumab and pertuzumab (10 μg/mL total antibody concentration) followed by a two hour incubation with Baby Rabbit complement (BCR, as described in Example 4) or with normal human serum (NETS) of three healthy donors (1:50 dilution, NETS 1, NETS 2, NETS 3). Human IgG1 with kappa light was used as isotype control.

Readout of cell lysis (LDH release) was performed on a Tecan sunrise reader using the LDH Cytotoxicity kit (Roche Diagnostics GmbH, Mannheim, Germany, Cat. No. 11644793001). Mean Lysis (in %) is the signal in relation to 3% Triton-X treated cells (maximum lysis). Experiment was performed in triplicates.

| | specific lysis [%] | | | |
|---|---|---|---|---|
| antibody/antibodies | BRC | NHS 1 | NHS 2 | NHS 3 |
| trastuzumab | 12.5 ± 0.3 | 0.6 ± 0.6 | −2.9 ± 0.5 | −2.3 ± 0.6 |
| pertuzumab | 16.5 ± 0.9 | −1.9 ± 1.2 | −3.8 ± 0.4 | −4.0 ± 0.7 |
| combination of | 46.9 ± 2.0 | 3.6 ± 1.5 | −0.6 ± 0.2 | −0.9 ± 1.5 |
| trastuzumab and | | | | |
| pertuzumab | | | | |
| human IgG1, kappa | 4.9 ± 1.2 | −3.5 ± 0.8 | −6.8 ± 1.7 | −5.2 ± 0.7 |

### Example 8

### siRNA Mediated Knockdown of CD55, CD59 and CD46

### Generation of Cell Lines

For the CD46, CD55 and CD59 knockdown, SK-OV-3 cells were treated with corresponding siRNA (Biospring; CD46 Cat. No. 203525-A, CD55 Cat. No. 203526-A, CD59 Cat. No. 203527-A), one control siRNA (Biospring, Cat. No. 203524-A) and the transfection reagent LipofectAmine (Invitrogen, Cat. No. 13778-100). The quantities used were according to the manufacturer's protocol. After three days of cultivation the amount of CD46, CD55 and CD59 on the cell surface was determined by FACS-analysis using a cell suspension with 1-2×10⁵ cells in 50 μL and master mix of FACS-antibodies. The antibody-master mix contained 1 μL each of anti-CD-55-APC antibody (BD Pharmingen, Cat. No. 555696) and anti-CD59-PE antibody (BD Pharmingen, Cat. No. 555764) and 10 μL of anti-CD46-FITC antibody (BD Pharmingen, Cat. No. 555949), 10% mouse serum (Southern Biotech, Cat. No. 0050-01) and FACS-Buffer (5 mL DPBS supplemented with 20 μL BSA). The FACS antibodies were titrated to determine the appropriate concentration to be employed. For isotype control, 20 μL IgG2a,k-FITC (BD Pharmingen, Cat. No. 556652), IgG2a,k-APC (BD Pharmingen, Cat. No. 552893), IgG2a,k-PE (BD Pharmingen, Cat. No. 551438) each with 10% mouse serum and FACS-Buffer were used. Cells were incubated with the above-mentioned FACS-antibodies for 30 minutes at 4° C. and 20 rpm, washed with 600 μL ice-cold DPBS buffer and resuspended in 200 μL Cytofix (BD Pharmingen, Cat. No. 554655). The FACS analysis was performed on a FACS Canto II.

| | | wild-type SK-OV-3 | knockdown SK-OV-3 |
|---|---|---|---|
| target | signal | cells | cells |
| CD46 | FITC | 683 | 662 |
| CD55 | APC | 1447 | 275 |
| CD59 | PE | 1192 | 649 |

A significant knockout was achieved for CD 55 (about 80% knockdown). The expression of CD 59 was down-regulated by about 45%. CD46 shows no change in the expression level.

CDC after Knockdown

For CD46, CD55 and CD59 knockdown, SK-OV-3 cells were treated with the corresponding siRNAs (Biospring; CD46 Cat. No. 203525-A, CD55 Cat. No. 203526-A, CD59 Cat. No. 203527-A) and the transfection reagent LipofectAmine (Invitrogen, Cat. No. 13778-100). The quantities used were according to the manufacturer's protocol. After three days of cultivation the amount of CD46, CD55 and CD59 on the cell surface was determined by FACS-analysis (see above). At day four a CDC-assay was performed with wild-type (=non-siRNA treated) SK-OV-3, SK-OV-3-triple cells (transfected with all three siRNAs) and SK-OV-3-Contrl.siRNA (transfected with an unspecific control siRNA). For the CDC-Assay 10.000 cells per well were seeded into a 96-well flat bottom plate (Thermo Scientific, Nunclon Delta Surface) containing 100 μL per well in AIM-V medium (Gibco, Cat. No. 0870112-DK) and were incubated for 20 hours at 37° C. and 5% CO₂. Thereafter trastuzumab, pertuzumab, human IgG1, kappa (Sigma, Cat. No. I5154) and bispecific anti-HER2 antibody (common light chain) were tested at a final concentration of 10 μg/mL. Triton-X (Roche Diagnostics GmbH, Mannheim, Germany, Cat. No. 11332481001) at a final concentration of 1% was used for the determination of the maximal lysis. All samples were incubated for 30 min. at 37° C. Subsequently, Baby Rabbit complement (BRC) (Cedarlane, Cat. No. CL3441) and Normal Human Serum (NETS) was added at a final dilution of 1/30 and the plates were incubated for 2 hours at 37° C. (final volume/well=150 μL). The amount of cell lysis was determined via LDH activity using the Cytotoxicity Detection Kit (Roche Diagnostics GmbH, Mannheim, Germany, Cat. No. 11644793001). The absorbance was determined at 490 nm and 620 nm using a Tecan Sunrise reader.

As positive control the following samples were used:
medium control: SK-OV-3 cells with AIM-V medium
spontaneous lysis: SK-OV-3 cells with active BRC
maximal lysis: SK-OV-3 cells with 1% Triton-X
isotype control: SK-OV-3 cells with 10 μg/mL human IgG, kappa and BRC
negative control: SK-OV-3 cells with 10 μg/mL antibody/composition and heat inactivated BRC
assay control: SK-OV-3 cells with 10 μg/mL trastuzumab and pertuzumab and active BRC.
   The results are shown in FIG. 4.

In the presence of NETS as source of complement the knockdown of CD55 and CD59 is absolutely required to exert CDC. The tedious siRNA knockdown procedure can be overcome by the use of BRC. The assay showed no influence by the presence of mCRPs on the carcinoma cells. This is the prerequisite for using the assay as reported herein for high throughput screening of different antibody formats (besides the screening for different antibody combinations) or plain as a positive control for other CDC assays.

The positive control showed that the CDC assay was working. The comparison of the OD 490/620 nm and the specific cytotoxicity (%) of SK-OV-3, SK-OV-3-triple-KO and SK-OV-3-Contrl.siRNA showed that the control siRNA does not induce cytotoxicity.

### Example 9

### CDC Assay by Manipulation of Membrane Bound Complement Regulatory Proteins (mCRPs)

To overcome restrictive factors produced by the target cells that could influence the assay, the amount of mCRPs, a group of proteins inhibiting different stages of the CDC process, on the target cells was decreased.

It has been found that the use of inhibitory anti-mCRP antibodies in order to block the mCRPs (CD46, CD55 and CD59) on tumor cells tremendously facilitates the CDC-assay. The currently established technology using siRNAs is very time-consuming due to the required number of pipetting steps. The herein reported new method is an important improvement for the evaluation of therapeutic antibodies in combination with NHS.

Determination of Dilution of the Blocking Anti-mCRP Antibodies to Saturate the Respective Cell Surface mCRPs

FACS analyses of titrated anti-mCRP mAbs was used to determine the dilution that is just sufficient for a saturated staining of the carcinoma cells. The optimal dilution showing a fluorescence signal relatively close to the max. fluorescence signal was defined as 1-time saturating concentration (1× conc. in the following Table).

| | | Stock | | Final | | |
|---|---|---|---|---|---|---|
| | | concen- | Dilu- | concen- | Geom. | Geom. |
| | | tration | tion | tration | Mean. | Mean. |
| Antibody | Host | [μg/ml] | 1/. . . | [μg/ml] | FITC | PE |
| CD46 #383 | mouse | 100 | 10 | 10 | 84035 | |
| CD46 #383 | mouse | 100 | 100 | 1 | 81769 | |
| CD46 #383 | mouse | 100 | 500 | 0.2 | 73953 | |
| CD46 #383 | mouse | 100 | 1000 | 0.1 | 50035 | |
| CD46 #383 | mouse | 100 | 5000 | 0.02 | 15768 | |
| CD46 #383 | mouse | 100 | 10000 | 0.01 | 13979 | |
| CD59 #384 | mouse | 1000 | 10 | 100 | 95245 | |
| CD59 #384 | mouse | 1000 | 100 | 10 | 95477 | |
| CD59 #384 | mouse | 1000 | 500 | 2 | 95479 | |
| CD59 #384 | mouse | 1000 | 1000 | 1 | 95474 | |
| CD59 #384 | mouse | 1000 | 5000 | 0.2 | 94007 | |
| CD59 #384 | mouse | 1000 | 10000 | 0.1 | 82389 | |
| CD59 #385 | rat | 1000 | 10 | 100 | | 55381 |
| CD59 #385 | rat | 1000 | 100 | 10 | | 64215 |
| CD59 #385 | rat | 1000 | 500 | 2 | | 62710 |
| CD59 #385 | rat | 1000 | 1000 | 1 | | 60516 |
| CD59 #385 | rat | 1000 | 5000 | 0.2 | | 53801 |
| CD59 #385 | rat | 1000 | 10000 | 0.1 | | 50067 |
| CD46 #386 | mouse | 1000 | 10 | 100 | 87561 | |
| CD46 #386 | mouse | 1000 | 100 | 10 | 79540 | |
| CD46 #386 | mouse | 1000 | 500 | 2 | 75514 | |
| CD46 #386 | mouse | 1000 | 1000 | 1 | 75220 | |
| CD46 #386 | mouse | 1000 | 5000 | 0.2 | 66388 | |
| CD46 #386 | mouse | 1000 | 10000 | 0.1 | 53534 | |
| CD55 #387 | mouse | 1000 | 10 | 100 | 31950 | |
| CD55 #387 | mouse | 1000 | 100 | 10 | 26893 | |
| CD55 #387 | mouse | 1000 | 500 | 2 | 25177 | |
| CD55 #387 | mouse | 1000 | 1000 | 1 | 25069 | |
| CD55 #387 | mouse | 1000 | 5000 | 0.2 | 23739 | |
| CD55 #387 | mouse | 1000 | 10000 | 0.1 | 24110 | |

| | | | Geom. | Geom. | | |
|---|---|---|---|---|---|---|
| | | Dilution | Mean. | Mean. | | |
| Antibody | Host | 1/. . . | FITC | PE | 1x conc. | 10x conc. |
| CD46 #383 | mouse | 100 | 81769 | | 1:300≈0.3 μg/ml | 3.0 μg/ml |
| CD59 #384 | mouse | 5000 | 94007 | | 1:5000≈0.2 μg/ml | 2.0 μg/ml |
| CD59 #385 | rat | 10000 | | 50067 | 1:10000≈0.1 μg/ml | 1.0 μg/ml |
| CD46 #386 | mouse | 5000 | 66388 | | 1:5000≈0.2 μg/ml | 2.0 μg/ml |
| CD55 #387 | mouse | 10000 | 24110 | | 1:10000≈0.1 μg/ml | 1.0 μg/ml |

Analysis of CDC using the “1-Times Saturating” Concentration of Blocking Anti-mCRP Antibodies

A CDC-assay with NHS and untreated SK-OV3 cells was performed with different combinations of inhibitory anti-mCRP mAbs at a 1-times saturating concentration in the presence of 10 μg/ml Per+Tra. It can be seen that single anti-mCRP antibodies have no significant impact on the CDC. The results are shown in FIG. 7 and the following Table.

| | | AB | Conc. | Com- | Aver | Aver |
|---|---|---|---|---|---|---|
| Samples | cells | 1 + 2 | mCRP | plement | (OD) | (% Lysis) |
| Medium | SK-OV3 | — | — | — | 0.108 | −7.55 |
| control | | | | | | |
| CD46 #383 | SK-OV3 | Per/Tra | 1x | 1/30 NHS | 0.235 | −0.47 |
| CD46 #383 | SK-OV3 | Per/Tra | 1x | 1/30 NHS | 0.364 | 6.64 |
| CD55 #387 | | | | | | |
| CD59 #384 | | | | | | |
| Spontaneous | SK-OV3 | — | — | 1/30 BRC | 0.329 | −1.09E−15 |
| lysis | | | | | | |
| CD46 #386 | SK-OV3 | Per/Tra | 1x | 1/30 NHS | 0.240 | −0.19 |
| CD46 #383 | SK-OV3 | Per/Tra | 1x | 1/30 NHS | 0.247 | 0.20 |
| CD55 #387 | | | | | | |
| CD59 #385 | | | | | | |
| Spontaneous | SK-OV3 | — | — | 1/30 NHS | 0.244 | −5.92E−16 |
| lysis | | | | | | |
| CD55 #387 | SK-OV3 | Per/Tra | 1x | 1/30 NHS | 0.248 | 0.22 |
| CD46 #386 | SK-OV3 | Per/Tra | 1x | 1/30 NHS | 0.422 | 9.86 |
| CD55 #387 | | | | | | |
| CD59 #384 | | | | | | |
| Isotype | SK-OV3 | IgG1 | — | 1/30 BRC | 0.343 | 0.80 |
| control | | | | | | |
| CD59 #384 | SK-OV3 | Per/Tra | 1x | 1/30 NHS | 0.245 | 0.09 |
| CD46 #386 | SK-OV3 | Per/Tra | 1x | 1/30 NHS | 0.266 | 1.22 |
| CD55 #387 | | | | | | |
| CD59 #385 | | | | | | |
| Isotype | SK-OV3 | IgG1 | — | 1/30 NHS | 0.283 | 2.19 |
| control | | | | | | |
| CD59 #385 | SK-OV3 | Per/Tra | 1x | 1/30 NHS | 0.251 | 0.37 |
| Max. Lysis | SK-OV3 | — | — | Trit-X | 2.051 | 100 |
| CD55 #387 | SK-OV3 | Per/Tra | 1x | 1/30 NHS | 0.431 | 10.3 |
| CD59 #384 | | | | | | |
| Per/Tra | SK-OV3 | Per/Tra | — | 1/30 BRC | 1.545 | 70.6 |
| CD55 #387 | SK-OV3 | Per/Tra | 1x | 1/30 NHS | 0.282 | 2.14 |
| CD59 #385 | | | | | | |
| Per/Tra | SK-OV3 | Per/Tra | — | 1/30 BRC | 0.160 | −9.81 |
| | | | | inactive | | |
| Per/Tra | SK-OV3 | Per/Tra | — | 1/30 NHS | 0.289 | −2.84 |
| Per/Tra | SK-OV3 | Per/Tra | — | 1/30 NHS | 0.133 | −6.14 |
| | | | | inactive | | |

Analysis of CDC using the “10×” Concentration of Blocking Anti-mCRP Antibodies

A CDC-assay with NHS and untreated SK-OV3 cells was performed with different combinations of inhibitory anti-mCRP mAbs at a 10-times saturating concentration in the presence of 10 μg/ml Per+Tra.

It can be seen that the 1-times saturating concentration of anti-mCRP blocking mAbs shows a much lower CDC-lysis than the 10-times saturating concentration of anti-mCRP mAb. The 10-times saturating concentration of anti-mCRP mAbs significantly enhances the CDC-lysis of tumor cells using NHS. Each inhibitory antibody combination of CD55 with CD59 as well as CD46 with CD55 and CD59 reaches a similar range of CDC-lysis (41%) using the Per+Tra antibody combination compared to the mCRP insensitive BRC. Single anti-mCRP antibodies have no significant impact on CDC (see FIG. 8 and the following Table).

| | | AB | mCRP | Conc. | Comple- | Aver | Aver |
|---|---|---|---|---|---|---|---|
| Samples | cells | 1 + 2 | 1 + 2 + 3 | mCRP | ment | (OD) | (% Lysis) |
| Medium | SK-OV3 | — | — | — | — | 0.100 | −7.1 |
| control | | | | | | | |
| CD46 #383 | SK-OV3 | Per/Tra | 383 | 10x | 1/30 NHS | 0.243 | 0.79 |
| CD46 #383 | SK-OV3 | Per/Tra | 383/387/384 | 10x | 1/30 NHS | 1.029 | 44.3 |
| CD55 #387 | | | | | | | |
| CD59 #384 | | | | | | | |
| Spontaneous | SK-OV3 | — | — | — | 1/30 BRC | 0.359 | 0 |
| lysis | | | | | | | |
| CD46 #386 | SK-OV3 | Per/Tra | 386 | 10x | 1/30 NHS | 0.241 | 0.62 |
| CD46 #383 | SK-OV3 | Per/Tra | 383/387/385 | 10x | 1/30 NHS | 0.819 | 32.7 |
| CD55 #387 | | | | | | | |
| CD59 #385 | | | | | | | |
| Spontaneous | SK-OV3 | — | — | — | 1/30 NHS | 0.228 | 5E−16 |
| lysis | | | | | | | |
| CD55 #387 | SK-OV3 | Per/Tra | 387 | 10x | 1/30 NHS | 0.243 | 0.81 |
| CD46 #386 | SK-OV3 | Per/Tra | 386/387/384 | 10x | 1/30 NHS | 0.771 | 30.1 |
| CD55 #387 | | | | | | | |
| CD59 #384 | | | | | | | |
| Isotype | SK-OV3 | IgG1 | — | — | 1/30 BRC | 0.355 | −0.26 |
| control | | | | | | | |
| CD59 #384 | SK-OV3 | Per/Tra | 384 | 10x | 1/30 NHS | 0.332 | 5.72 |
| CD46 #386 | SK-OV3 | Per/Tra | 386/387/385 | 10x | 1/30 NHS | 0.926 | 38.6 |
| CD55 #387 | | | | | | | |
| CD59 #385 | | | | | | | |
| Isotype | SK-OV3 | IgG1 | — | — | 1/30 NHS | 0.304 | 4.17 |
| control | | | | | | | |
| CD59 #385 | SK-OV3 | Per/Tra | 385 | 10x | 1/30 NHS | 0.335 | 5.89 |
| Max. Lysis | SK-OV3 | — | — | — | Trit-X | 2.034 | 100 |
| CD55 #387 | SK-OV3 | Per/Tra | 387/384 | 10x | 1/30 NHS | 0.823 | 33.0 |
| CD59 #384 | | | | | | | |
| Per/Tra | SK-OV3 | Per/Tra | — | — | 1/30 BRC | 1.051 | 41.3 |
| CD55 #387 | SK-OV3 | Per/Tra | 387/385 | 10x | 1/30 NHS | 0.939 | 39.4 |
| CD59 #385 | | | | | | | |
| Per/Tra | SK-OV3 | Per/Tra | — | — | 1/30 BRC | 0.268 | −5.46 |
| | | | | | inactive | | |
| Per/Tra | SK-OV3 | Per/Tra | — | — | 1/30 NHS | 0.280 | 2.88 |
| Per/Tra | SK-OV3 | Per/Tra | — | — | 1/30 NHS | 0.257 | 1.55 |
| | | | | | inactive | | |

Comparative Analysis of CDC using the “10×” Saturating Concentration of Blocking Anti-mCRP Antibodies or mCRP siRNAs

The following NHS CDC-assay was carried out to compare both approaches (siRNA and mCRP-blocking mAbs) in the effect of blocking the CDC-inhibiting mCRP function.

SK-OV3, SK-OV3(Ctrl-siRNA) or SK-OV3(Triple-KO) cells were used in combination with 10 μg/ml Per+Tra and different anti-mCRP mAb combinations using the 10-times saturating concentration.

In this comparative assays the triple combination of functional anti-mCRP mAbs using untreated SK-OV3 cells and 10 μg/ml Per+Tra leads to the same level of CDC (66% lysis) as the siRNA treated SK-OV3(Triple-KO) cells (64% lysis). Interestingly, only the combination of all three anti-mCRP mAbs induce CDC at the same level as transfection with the respective mCRP siRNAs. The results are shown in FIG. 9 and the following Table.

| | | AB | mCRP | Comple- | Aver | Aver |
|---|---|---|---|---|---|---|
| Samples | cells | 1 + 2 | 1 + 2 + 3 | ment | (OD) | (% Lysis) |
| Medium | SK-OV3 | — | — | — | 0.096 | −8.22 |
| Control | | | | | | |
| CD46 #383 | SK-OV3 | Per + Tra | CD46 #383 | 1/30 NHS | 0.251 | 1.10 |
| CD46 #383 | SK-OV3 | Per + Tra | CD46 #383 | 1/30 NHS | 1.062 | 83.2 |
| | (Triple-KO) | | | | | |
| CD46 #383 | SK-OV3 | Per + Tra | CD46 #383 | 1/30 NHS | 0.409 | 4.14 |
| | (Ctrl-siRNA) | | | | | |
| Spontaneous | SK-OV3 | — | — | 1/30 NHS | 0.232 | 3.47E−17 |
| Lysis | | | | | | |
| CD55 #387 | SK-OV3 | Per + Tra | CD55 #387 | 1/30 NHS | 0.256 | 1.40 |
| CD55 #387 | SK-OV3 | Per + Tra | CD55 #387 | 1/30 NHS | 1.029 | 79.1 |
| | (Triple-KO) | | | | | |
| CD55 #387 | SK-OV3 | Per + Tra | CD55 #387 | 1/30 NHS | 0.397 | 3.00 |
| | (Ctrl-siRNA) | | | | | |
| Max. Lysis | SK-OV3 | — | — | Trit-X | 1.895 | 100 |
| CD59 #384 | SK-OV3 | Per + Tra | CD59 #384 | 1/30 NHS | 0.368 | 8.14 |
| CD59 #384 | SK-OV3 | Per + Tra | CD59 #384 | 1/30 NHS | 1.094 | 87.2 |
| | (Triple-KO) | | | | | |
| CD59 #384 | SK-OV3 | Per + Tra | CD59 #384 | 1/30 NHS | 0.453 | 8.38 |
| | (Ctrl-siRNA) | | | | | |
| Spontaneous | SK-OV3 | — | — | 1/30 NHS | 0.386 | −2.27E−15 |
| Lysis | (Triple-KO) | | | | | |
| CD55 #387 | SK-OV3 | Per + Tra | CD55 #387 + | 1/30 NHS | 0.641 | 24.6 |
| CD59 #384 | | | CD59 #384 | | | |
| CD55 #387 | SK-OV3 | Per + Tra | CD55 #387 + | 1/30 NHS | 1.101 | 88.0 |
| CD59 #384 | (Triple-KO) | | CD59 #384 | | | |
| CD55 #387 | SK-OV3 | Per + Tra | CD55 #387 + | 1/30 NHS | 0.538 | 16.5 |
| CD59 #384 | (Ctrl-siRNA) | | CD59 #384 | | | |
| Max. Lysis | SK-OV3 | — | — | Trit-X | 1.199 | 100 |
| | (Triple-KO) | | | | | |
| CD46 #383 | SK-OV3 | Per + Tra | CD46 #383 + | 1/30 NHS | 1.333 | 66.2 |
| CD55 #387 | | | CD55 #387 + | | | |
| CD59 #384 | | | CD59 #384 | | | |
| CD46 #383 | SK-OV3 | Per + Tra | CD46 #383 + | 1/30 NHS | 1.223 | 103.0 |
| CD55 #387 | (Triple-KO) | | CD55 #387 + | | | |
| CD59 #384 | | | CD59 #384 | | | |
| CD46 #383 | SK-OV3 | Per + Tra | CD46 #383 + | 1/30 NHS | 0.581 | 20.6 |
| CD55 #387 | (Ctrl-siRNA) | | CD55 #387 + | | | |
| CD59 #384 | | | CD59 #384 | | | |
| Spontaneous | SK-OV3 | — | — | 1/30 NHS | 0.366 | 0 |
| Lysis | (Ctrl-siRNA) | | | | | |
| Per/Tra | SK-OV3 | Per + Tra | — | 1/30 NHS | 0.260 | 1.64 |
| Per/Tra | SK-OV3 | Per + Tra | — | 1/30 NHS | 0.907 | 64.1 |
| | (Triple-KO) | | | | | |
| Per/Tra | SK-OV3 | Per + Tra | — | 1/30 NHS | 0.419 | 5.11 |
| | (Ctrl-siRNA) | | | | | |
| Max. Lysis | SK-OV3 | — | — | Trit-X | 1.411 | 100 |
| | (Ctrl-siRNA) | | | | | |
| Isotype | SK-OV3 | IgG | — | 1/30 NHS | 0.270 | 2.30 |
| Control | | | | | | |
| Isotype | SK-OV3 | IgG | — | 1/30 NHS | 0.426 | 4.97 |
| Control | (Triple-KO) | | | | | |
| Isotype | SK-OV3 | IgG | — | 1/30 NHS | 0.413 | 4.54 |
| Control | (Ctrl-siRNA) | | | | | |

Comparative Analysis of CDC using the “10×” Saturating Concentration of Blocking Anti-mCRP Antibodies or mCRP siRNAs w/o Per and Tra

The following NHS CDC-assay was carried out to evaluate the impact of the murine functional Fc-region (anti-mCRP antibodies) on the CDC level in a single experiment with the respective siRNA approach.

SK-OV3, SK-OV3(Ctrl-siRNA) or SK-OV3(Triple-KO) cells were used in combination with different anti-mCRP mAb combinations of mCRP antibodies using the 10-times saturating concentration.

This control CDC assay confirms that murine anti-mCRP mAbs per se without Per+Tra are not able to induce CDC with NHS. Interestingly the human complement components of the NHS interact highly species specific exclusively with the human Per and Tra IgG to elicit CDC and do not interact with the murine IgGs despite the fact that the carcinoma cells are saturated with 3 different murine IgG molecules. The results are shown in the following Table and FIG. 10.

| | | AB | mCRP | Comple- | Aver | Aver |
|---|---|---|---|---|---|---|
| Samples | cells | 1 + 2 | 1 + 2 + 3 | ment | (OD) | (% Lysis) |
| Medium | SK-OV3 | — | — | — | 0.112 | −6.30 |
| Control | | | | | | |
| CD46 #383 | SK-OV3 | — | CD46 #383 | 1/30 | 0.236 | 0.83 |
| | | | | NHS | | |
| CD46 #383 | SK-OV3 | — | CD46 #383 | 1/30 | 0.341 | 0.88 |
| | (Triple-KO) | | | NHS | | |
| CD46 #383 | SK-OV3 | — | CD46 #383 | 1/30 | 0.365 | −8.71 |
| | (Ctrl-siRNA) | | | NHS | | |
| Spontaneous | SK-OV3 | — | — | 1/30 | 0.221 | 5.55E−16 |
| Lysis | | | | NHS | | |
| CD55 #387 | SK-OV3 | — | CD55 #387 | 1/30 | 0.254 | 1.90 |
| | | | | NHS | | |
| CD55 #387 | SK-OV3 | — | CD55 #387 | 1/30 | 0.362 | 3.56 |
| | (Triple-KO) | | | NHS | | |
| CD55 #387 | SK-OV3 | — | CD55 #387 | 1/30 | 0.395 | −6.08 |
| | (Ctrl-siRNA) | | | NHS | | |
| Max. Lysis | SK-OV3 | — | — | Trit-X | 1.954 | 100 |
| CD59 #384 | SK-OV3 | — | CD59 #384 | 1/30 | 0.289 | 3.87 |
| | | | | NHS | | |
| CD59 #384 | SK-OV3 | — | CD59 #384 | 1/30 | 0.384 | 6.27 |
| | (Triple-KO) | | | NHS | | |
| CD59 #384 | SK-OV3 | — | CD59 #384 | 1/30 | 0.408 | −4.93 |
| | (Ctrl-siRNA) | | | NHS | | |
| Spontaneous | SK-OV3 | — | — | 1/30 | 0.334 | 2.37E−15 |
| Lysis | (Triple-KO) | | | NHS | | |
| CD55 #387 | SK-OV3 | — | CD55 #387 + | 1/30 | 0.488 | 15.41 |
| CD59 #384 | | | CD59 #384 | NHS | | |
| CD55 #387 | SK-OV3 | | CD55 #387 + | 1/30 | 0.385 | 6.39 |
| CD59 #384 | (Triple-KO) | | CD59 #384 | NHS | | |
| CD55 #387 | SK-OV3 | — | CD55 #387 + | 1/30 | 0.519 | 4.85 |
| CD59 #384 | (Ctrl-siRNA) | | CD59 #384 | NHS | | |
| Max. Lysis | SK-OV3 | — | — | Trit-X | 1.135 | 100 |
| | (Triple-KO) | | | | | |
| CD46 #383 | SK-OV3 | — | CD46 #383 + | 1/30 | 0.322 | 5.78 |
| CD55 #387 | | | CD55 #387 + | NHS | | |
| CD59 #384 | | | CD59 #384 | | | |
| CD46 #383 | SK-OV3 | — | CD46 #383 + | 1/30 | 0.359 | 3.09 |
| CD55 #387 | (Triple-KO) | | CD55 #387 + | NHS | | |
| CD59 #384 | | | CD59 #384 | | | |
| CD46 #383 | SK-OV3 | — | CD46 #383 + | 1/30 | 0.562 | 8.60 |
| CD55 #387 | (Ctrl-siRNA) | | CD55 #387 + | NHS | | |
| CD59 #384 | | | CD59 #384 | | | |
| Spontaneous | SK-OV3 | — | — | 1/30 | 0.464 | 1.62E−15 |
| Lysis | (Ctrl-siRNA) | | | NHS | | |
| Per/Tra | SK-OV3 | Per + Tra | — | 1/30 | 0.293 | 4.12 |
| | | | | NHS | | |
| Per/Tra | SK-OV3 | Per + Tra | — | 1/30 | 1.130 | 99.3 |
| | (Triple-KO) | | | NHS | | |
| Per/Tra | SK-OV3 | Per + Tra | — | 1/30 | 0.455 | −0.76 |
| | (Ctrl-siRNA) | | | NHS | | |
| Max. Lysis | SK-OV3 | — | — | Trit-X | 1.603 | 100 |
| | (Ctrl-siRNA) | | | | | |
| Per/Tra | SK-OV3 | Per + Tra | — | 1/30 | 0.185 | −2.11 |
| | | | | NHS | | |
| | | | | (inactive) | | |
| Per/Tra | SK-OV3 | Per + Tra | — | 1/30 | 0.294 | −4.97 |
| | (Triple-KO) | | | NHS | | |
| | | | | (inactive) | | |
| Per/Tra | SK-OV3 | Per + Tra | — | 1/30 | 0.356 | −9.46 |
| | (Ctrl-siRNA) | | | NHS | | |
| | | | | (inactive) | | |

These results demonstrate that it is possible to avoid the laborious and time-consuming siRNA approach to overcome the CDC inhibition by the mCRPs in the human system by using the 10-times saturating concentration of functional anti-mCRP mAb s.

### General Methods

CDC-Assay with Baby Rabbit Complement

This assay was performed to determine the amount of lysed cells resulting from the antibody-driven complement dependent cytotoxicity.

On the first day SK-OV3 cells were seeded with a concentration of 1*10E04 cells per well in 100 μl growing medium into a 96-well flat-bottom plate (Thermo Scientific) and incubated for about 20 h at 37° C. and 5% CO₂. The following day antibody-stock solutions (e.g. Per+Tra) were diluted with AIM-V serum free medium (Gibco) to the desired concentration, which was mostly 5 or 10 μg/ml.

To obtain maximal cell lysis, 10% of the detergent Triton-X 100 (Roche Diagnostics GmbH) was diluted with AIM-V serum free medium, resulting in a final concentration of 1% per well.

After washing the cells once with 100 μl AIM-V serum free medium, 50 μl of AIM-V serum free medium was added to each well. Afterwards 50 μl antibody or Triton-X 100 stock solution were added respectively. The remaining wells were filled with 50 μl of AIM-V serum free medium. Then the cells were incubated for 30 min at 37° C. and 5% CO₂.

Just before the incubation period ended, the baby rabbit complement dilutions were prepared. The BRC lyophilisate (Biozol) was dissolved 1/10 in AIM-V serum free medium. The complement dilutions were stored on ice for 15 min maximum until usage. As negative control of the complement, an aliquot of the diluted BRC was incubated for 30 min at 59° C. in a water bath. Activated and inactivated complement dilutions (50 μl each) were added to the corresponding wells. This resulted in a final dilution of 1/30 BRC per well. After adding the complement, the plate was incubated for 2 h at 37° C. and 5% CO₂. Subsequently, the plate was centrifuged for 10 min at 200 g and 50 μl supernatant was transferred into a clean 96-well flat-bottom plate. Lactate dehydrogenase (LDH) reaction mix (Roche Diagnostics GmbH) was prepared according to the manufacture's instruction. To each well 50 μl reaction mix was added and incubated for 15 min at 37° C. and 5% CO₂. An enzymatic reaction was performed to determine the LDH activity, which equals the amount of dead cells. In this reaction tetrazolium salt was reduced to formazan. Formazan is a water soluble molecule and has an absorption maximum at about 500 nm. The amount of formazan correlates to the number of lyzed cells and thus with the LDH activity of the culture supernatant.

The optical density (OD) was photometric measured with Infinite M1000 Pro Reader (Tecan) at 490 nm. The absorption at 620 nm was determined as reference. The calculated difference between measurement and reference measurement was evaluated with Spotfire 6.5.3 software (TIBCO).

Each sample was performed as a triplicate. In general the following samples were measured:

Medium control: In these wells the cells were only treated with AIM-V serum free medium to determine the effect of the medium on CDC. The value of this sample served as background measurement.

Spontaneous lysis: In these wells the cells were only treated with BRC without any antibodies to determine the effect of the complement on cells. This sample served as control for spontaneous lysis.

Max. lysis: In these wells the cells were treated with 1% Triton-X to determine the maximal detectable release of LDH. This sample served as measurement for the max. lysis.

Antibodies with active complement: In these wells the cells were treated with different antibodies and active BRC to determine the specific induced effect of antibodies on CDC.

Antibodies with inactive complement: In these wells the cells were treated with antibodies and inactive BRC. This sample served as a control to see an effect of the antibodies without the presence of active complement.

In FIG. 5 exemplary settings of a standard CDC-assay, which served as positive control in case other antibodies than Per+Tra were used, is depicted.

The induced cytotoxicity was calculated by geometric means of the OD of triplicates as follows:

$$Specific Cytotoxicity [ \% ] = \frac{sample OD - spontaneuos lysis OD}{max lysis OD - spontaneuos lysis OD} * 100$$

In FIG. 6 the evaluation of the CDC-assay after the conversion of the OD values to the specific cytotoxicity is shown.

Max. lysis is always set to 100% and spontaneous lysis is always set to 0%.

siRNA Transfection

In order to knock down mCRPs, a siRNA transfection was performed as described below.

On the first day (d=0) cells were seeded into a 6-well flat-bottom plate with a concentration of about 2-3*10E05 cells/ml in order to achieve a confluence of 60-80% at the time of transfection. On the next day (d=1) 150 μl Opti-MEM medium (Gibco) was mixed with 9 μl LipofectAmine RNAi MAX reagent (Invitrogen). In the next step 9 μl siRNA (Biospring) with a concentration of 10 μM was mixed with 150 μl Opti-MEM medium. After this step, both dilutions were added 1:1 into a reaction vial and incubated for 5 minutes at room temperature. After the incubation, 250 μl of this mix was added to one 6-well. The effect of the transfection can be visualized and analyzed after 3 days by using FACS. The cells were transfected with a combination of CD46, CD55 and CD59 siRNA or with Ctrl-siRNA for this master thesis.

FACS Staining to Analyze siRNA Transfection

A FACS staining was performed to evaluate the effect siRNA transfection. At first, cell suspensions with 2*10E05 cells/50 μl of non-transfected cells, CD46, CD55 and CD59 siRNA transfected cells and Ctrl-siRNA transfected cells were prepared. Afterwards, the cells were stained with 50 μl direct labeled antibodies against the mCRPs as well as with the corresponding isotype controls (IC). The antibodies used for this staining are shown in the following Table.

| | Antibody | | | |
|---|---|---|---|---|
| Antibody | against | Host | Label | Isotype |
| #362 | CD59, | mouse | PE | IgG2a, k |
| | human | | | |
| #363 | CD55, | mouse | APC | IgG2a, k |
| | human | | | |
| #364 | CD46, | mouse | FITC | IgG2a, k |
| | human | | | |
| #365 | IC | mouse | FITC | IgG2a, k |
| #367 | IC | mouse | PE | IgG2a, k |
| #368 | IC | mouse | APC | IgG2a, k |

APC: Allophycocyanin
FITC: Fluorescein isothiocyanate
PE: Phycoerythrin
IC: Isotype control

The cells were labeled for 30 min on ice and afterwards washed twice with 200 μl ice-cold DPBS and centrifuged at 350 g for 5 min. The supernatant was removed and the pellet was resuspended with 150 μl Cytofix. Subsequently, a FACS analysis was performed by using the MACSQuant device. The tumor cells were analyzed with the following conditions as shown in the following Table.

| Cell treatment | Staining (#Antibody) |
|---|---|
| untreated | unstained |
| untreated | anti-CD46-FITC (#364) |
| untreated | anti-CD55-APC (#363) |
| untreated | anti-CD59-PE (#362) |
| untreated | anti-CD46-FITC (#364) anti-CD55-APC (#363) |
| | anti-CD59-PE (#362) |
| CD46, CD55 and CD59 | anti-CD46-FITC (#364) anti-CD55-APC (#363) |
| siRNA | anti-CD59-PE (#362) |
| CD46, CD55 and CD59 | IC-FITC (#365) IC-APC (#368) IC-PE (#367) |
| siRNA | |
| Ctrl-siRNA | anti-CD46-FITC (#364) anti-CD55-APC (#363) |
| | anti-CD59-PE (#362) |

CDC-Assay with Normal Human Serum after siRNA Transfection

After performing siRNA transfection and thus knocking down mCRPs successfully, the cells were able to be lysed using NHS. The assay was performed as described herein. Instead of using 1/30 BRC the assay was performed with 1/30 NHS. The NHS was previously produced and stored thereafter at −80° C.

FACS Titration of Inhibitory Anti-mCRP mAbs

This experiment was performed to standardize the concentration of mCRP-blocking, anti-mCRP mAbs for the use in further CDC-assays.

A cell suspension with 1*10E05 cells/50 μl was prepared and incubated with 50 μl of different antibody concentrations (from 100 ng/ml to 10 μg/ml) for 30 min on ice. Before adding an appropriate secondary antibody, the cells were washed twice with 200 μl ice-cold DPBS and centrifuged at 350 g for 5 min. After the incubation with the secondary antibody, the cells were washed again twice as described above. Afterwards, the cells were resuspended in 150 μl Cytofix and a FACS analysis was performed.

| | Antibody | | | |
|---|---|---|---|---|
| Antibody | against | Host | Label | Isotype |
| #255 | IgG, human | mouse | APC | mAb, IgG1k |
| #326 | IgG Fc, | goat | FITC | pAb |
| | rabbit | | | |

Blocking of mCRPs with Inhibitory Anti-mCRP mAbs

An alternative approach for the blocking of mCRPs was implemented in order to avoid laborious and extremely time-consuming work with siRNAs.

Inhibitory anti-mCRP mAbs were investigated (Christiansen et al., 1996; Harris et al., 1997; Sirena et al., 2004).

| | Antibody | | | | |
|---|---|---|---|---|---|
| Antibody | against | Host | Label | Isotype | Clone |
| #383 | CD46, | mouse | — | IgG1 | M177 |
| | human | | | | |
| #384 | CD59, | mouse | — | IgG2a | MEM43 |
| | human | | | | |
| #385 | CD59, | rat | — | IgG2b | YTH53.1 |
| | human | | | | |
| #386 | CD46, | mouse | — | IgG1 | MEM258 |
| | human | | | | |
| #387 | CD55, | mouse | — | IgG1 | BRIC216 |
| | human | | | | |

For staining standardization the optimal concentration of the inhibitory anti-mCRP mAbs was determined by titration using FACS. The staining was performed as described in the following way.

Approximately 1-3*10E05 cells/50 μl cells were stained with different anti-mCRP mAbs concentrations (100 ng/ml, 200 ng/ml, 1 μg/ml, 2 μg/ml, 10 μg/ml and 20 μg/ml). After washing twice with ice-cold DPBS, the cells were stained with an appropriate secondary mAbs dependent on the isotype of the primary antibody. After two more washing steps, stained cells were resuspended with 150 μl Cytofix. Subsequently, a FACS-analysis was performed by using the MACSQuant device.

A standardized antibody concentration was determined by analyzing the FACS dot plots (data not shown). The dilution for the antibodies (one-fold saturating concentration) was set to the dilution value just showing a saturated staining on the tumor cells.

After the one-fold concentration was identified, the optimal compilation of five anti-mCRP mAbs was analyzed by performing CDC-assays with NHS. Single anti-mCRP mAbs, mAb pairs (only for CD55 and CD59) and triple mAb combinations were used. The inhibitory anti-mCRP mAbs were added simultaneously with therapeutic antibodies (e.g. Per+Tra). By adding the blocking anti-mCRP mAbs, as a further ingredient in the CDC-assay, the total assay-volume increased from 150 μl to 200 μl. The concentration of stock solutions of all other ingredients (e.g. Trit-X and BRC or NHS) was adjusted accordingly to allow for a max. volume of 200 μl.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for determining complement dependent cytotoxicity of a composition wherein the composition comprises
i) a first binding site that specifically binds to a first epitope on a first antigen, which is conjugated to a first Fc-region polypeptide of human origin, and
ii) a second binding site that specifically binds to a second epitope on the first antigen or on a second antigen, which is conjugated to a second Fc-region polypeptide of human origin,
wherein the method comprises the following steps:
a) incubating a human carcinoma cell of epithelial origin expressing the first antigen or the first antigen and the second antigen with the composition and a mixture of anti-mCRP antibodies,
b) adding normal human serum or rabbit complement to the mixture of a), and
c) determining cell lysis and thereby determining complement dependent cytotoxicity of the composition.

2. The method according to claim 1, wherein the mixture of anti-mCRP antibodies is a mixture comprising an anti-CD46 antibody, an anti-CD55 antibody and an anti-CD59 antibody.

3. The method according to claim 1, wherein the anti-mCRP antibodies have a non-human Fc-region.

4. The method according to claim 3, wherein the anti-mCRP antibodies have a murine Fc-region.

5. The method according to claim 1, wherein the anti-mCRP antibodies are added at a 10-times saturating concentration, whereby the 1-times saturating concentration is defined as the concentration determined by FACS analyses that is sufficient for a saturated staining of the cells.

6. The method according to claim 1, wherein the composition comprises a first human or humanized antibody that specifically binds to a first epitope on a first antigen and a second human or humanized antibody that specifically binds to a second epitope on a second antigen.

7. The method according to claim 1, wherein the composition comprises a human or humanized bispecific antibody that specifically binds to a first epitope on a first antigen and a second epitope on a second antigen.

8. The method according to claim 1, wherein the composition binds to a first epitope on the first antigen and a second epitope on the first antigen and the first epitope and the second epitope are different.

9. The method according to claim 8, wherein the first epitope and the second epitope are non-overlapping epitopes.

10. The method according to claim 1, wherein cell lysis is determined between 0.5 and 3 hours after the addition of complement.

11. The method according to claim 1, wherein the human carcinoma cell of epithelial origin is selected from the group consisting of human ovary adenocarcinoma cells, and human breast adenocarcinoma cells.

12. The method according to claim 1, wherein the human carcinoma cell of epithelial origin is selected from a SK-OV3 cell, and a MCF7 cell.

13. The method according to claim 1, wherein the rabbit complement is Baby Rabbit complement.

14. The method according to claim 1, wherein the ratio of the first binding site to the second binding site is of from 0.5:1 to 1:0.5.
